(19) United States
(12) Patent Application Publication
(10) Pub. No.: US 2011/0039775 A1
(43) Pub. Date: Feb. 17, 2011

(54) THYROID STIMULATING HORMONE FUSION PROTEINS

(76) Inventors: Peter Artymiuk, Sheffield (GB); Richard Ross, Sheffield (GB)

Correspondence Address: KLARQUIST SPARKMAN, LLP, 121 SW SALMON STREET, SUITE 1600, PORTLAND, OR, 97204, US

(21) Appl. No.: 12/933,386

(22) PCT Filed: Mar. 19, 2009
(86) PCT No.: PCT/GB2009/000742

§ 371 Date: Oct. 28, 2010

Related U.S. Application Data
(60) Provisional application No. 61/038,540, filed on Mar. 21, 2008.

(30) Foreign Application Priority Data
May 9, 2008 (GB) 0808402.2

(51) Int. Cl. A61K 38/24; C07K 14/59; C07H 21/00; C12N 15/63; C12N 5/10; A61P 5/14; A61P 35/00; A61P 19/10

(52) U.S. Cl. 514/9.7; 530/399; 536/23.4; 435/320.1; 435/325

(57) ABSTRACT

We disclose TSH fusion proteins comprising TSHα and/or TSH β and methods to treat diseases that would benefit from administration of TSH agonists and antagonists.

The invention relates to thyroid stimulating hormone [TSH] fusion proteins; dimers comprising said fusion protein; and methods to treat diseases that would benefit from administration of said fusion proteins.

There are many examples of bioactive polypeptides that function to regulate biological processes. For example thyrotrophin releasing hormone (TRH) from the hypothalamus regulates the release of thyroid stimulating hormone (TSH) from the pituitary which stimulates the release of thyroid hormones from the thyroid gland. TSH binds to the TSH receptor a GPCR resulting in increased uptake of iodine into the thyroid, thyroid growth and release of the thyroid hormones, thyroxine and trioiodothyronine. Deficiency in TSH due to hypopituitarism results in hypothyroidism and excess TSH from a TSH secreting pituitary tumour in hyperthyroidism. Hyperthyroidism or thyrotoxicosis may be caused by autoantibodies that stimulate the TSH receptor, Graves's disease. Another common cause of thyrotoxicosis is a multinodular goitre where the gland come autonomous, secretes excess thyroid hormones and consequently suppresses TSH levels. This also occurs in patients who have thyrotoxicosis due to a single toxic thyroid nodule. Thyrotoxicosis due to excess TSH secretion from pituitary tumours, a TSHoma, is rare. These tumours are difficult to treat and patients may have uncontrolled thyrotoxicosis. Hypothyroidism is most commonly caused by failure of the thyroid, primary hypothyroidism, where TSH levels are high. Less commonly hypothyrodism is related to TSH deficiency from the pituitary, secondary hypothyroidism.

Thyroid cancers occur at all ages. The primary treatment is surgery with complete removal of the thyroid gland, however almost invariably a small thyroid remnant is left after surgery. The usual practice is to ablate the thyroid remnant with radioactive iodine. Normal thyroid tissue and some thyroid cancers avidly take up radioactive iodine which then results in ablation of the tissue. Following ablation of the thyroid remnant then radioactive iodine can be used to screen patients for recurrence of their thyroid cancer. If patients have a thyroid cancer that takes up radioactive iodine then radioactive iodine can be used as therapy to treat the cancer.

Iodine uptake by the thyroid as well as other isotopes such as technetium is dependant on the presence TSH activity to stimulate their uptake into the thyroid. There is therefore a problem in stimulating uptake of isotopes of iodine or related molecules in situations where TSH levels are suppressed. This occurs in patients who are thyrotoxic for example with a multinodular goitre and those receiving thyroid hormone replacement after surgical treatment for thyroid cancer. It is generally considered that TSH may stimulate thyroid cancer regrowth thus patients who have undergone thyroidectomy for a thyroid cancer receive suppressive doses of thyroid hormone either T4 or T3. On these suppressive doses TSH is suppressed and it is not possible to undertake iodine or technetium or any isotope scanning of the thyroid. Before the availability of recombinant TSH patients would have to stop thyroid replacement to allow TSH levels to rise and as a consequence became frankly and symptomatically hypothyroid prior to scanning. This was unpleasant, unpredictable, and time consuming. Now with recombinant TSH patients can remain on thyroid hormone treatment during iodine scanning as they will be treated with recombinant TSH which will increase uptake of iodine and related molecules into the thyroid. This approach of recombinant TSH treatment can be used to increase uptake of iodine or related molecules into thyroid remnants, thyroid cancers, and multinodular goitres or any tumour or cancer that takes up iodine or related compounds. Current recombinant TSH has a relatively short half-life and therefore at least two doses are required on separate days prior to iodine treatment. A long acting TSH would be an advantage if it increased radioactive iodine isotopes or other isotopes uptake into the thyroid, a thyroid cancer or other cancer.

TSH agonists can be used to increase iodine, radioactive iodine, technetium and other isotopes of molecules uptake into the thyroid. This is of value in any situation where TSH levels are suppressed or even not elevated such as patients taking thyroid hormone, T4 and/or T3 or where patients have thyrotoxicosis due to a single toxic thyroid nodule, or multinodular goitre. In Graves disease patients generally have TSH stimulating antibodies however it maybe possible that a temporary treatment with TSH at the time of radioactive iodine therapy may selectively enhance iodine uptake into the thyroid and improve efficacy of treatment.

Another use for a TSH agonists would be in the treatment of secondary hypothyroidism where TSH levels are low. These patients usually take daily thyroxine therapy. A TSH agonist could potentially provide therapy if it could maintain thyroid hormone levels in the normal range. An advantage of this therapy could be maintenance of more physiological levels of thyroid hormones as currently most patients only receive thyroxine (T4) which is a prodrug for triiodothyronine (T3). TSH treatment would have the advantage of stimulating the physiological ratio of T4 and T3 from the thyroid where the thyroid is normally responsible for 20% of circulating T3 and 100% of T4. There is some evidence that TSH itself may be important in maintaining the integrity of other tissues such as bone and this could be an advantage of TSH therapy. Upto 50% of patients with hypothyroidism have a poor health related quality of life and potentially TSH therapy where appropriate maybe be of benefit.

TSH receptor antagonists could be used in situations where TSH levels are elevated and causing thyrotoxicosis or in situations where the TSH receptor is being activated by other molecules such as TSH receptor activating antibodies in Graves's disease. Thyrotoxicosis due to high TSH levels is uncommon but difficult to treat and due to TSHomas of the pituitary. A TSH receptor antagonist could be of value to control the thyroid in patients with TSH excess or TSH receptor activation. In Graves's disease the usual approach is to block thyroid hormone release form the thyroid using drugs such as carbimazole and propythiouracil. A TSH receptor antagonist could be used to treat Graves by directly preventing the TSH receptor antibodies activating the TSH receptor. This may have the added advantage of reducing the recurrence of Graves which occurs in 50% of patients. It has been considered that if you could reduce the antigen exposure in Graves, and the antigen is considered to be the TSH receptor, then you might reduce recurrence of Graves's disease. Complications of Graves's diseases include Graves's opthalamopathy with propotosis and damage to the eyes, pretibial myxoedema, osteopaenia and osteoporosis, myopathy, cardiomyopathy, atrial fibrillation, and dementia. Some if not all of these maybe related to expression of TSH receptor in other tissues such as intraorbital fat and muscle of the eye. In the situation of complications of Graves's disease a TSH receptor antagonist could prevent or reduce the complications of Graves's disease specifically thyroid associated opthalmopathy. The foetus in utero or newborn baby maybe at risk of neonatal thyrotoxicosis if the mother has Graves's diseases or has been treated for Graves's disease. The Graves antibodies can cross the placenta and cause thyrotoxicosis in the foetus or infant. A TSH receptor antagonist could be a therapy for neonatal thyrotoxicosis.

A problem associated with therapeutic peptides and therapeutic polypeptides is that the subject administered the peptide/polypeptide agent clears the molecule in some cases in a matter of minutes. This is due in part to proteolytic degradation and renal filtration. In many cases this is addressed by providing a combined formulation of peptide agent with a second agent that modulates the release of the peptide after administration.

The present disclosure addresses the problem of maintaining systemic levels of polypeptide therapeutics by reducing loss via proteolytic cleavage, renal filtration or any other means by which a pharmaceutically effective amount of polypeptide are reduced.

According to an aspect of the invention there is provided a fusion protein comprising Thyroid Stimulating Hormone α [TSHα] and/or Thyroid Stimulating Hormone β [TSHβ] wherein TSHα and/or TSHβ is optionally linked, either directly or indirectly, to the binding domain of TSH receptor.

In a preferred embodiment of the invention said fusion protein is an agonist.

In an alternative preferred embodiment of the invention said fusion protein is an antagonist.

In a preferred embodiment of the invention TSHα and/or TSHβ is linked to said receptor binding domain and is positioned amino terminal to said receptor binding domain in said fusion protein.

In an alternative preferred embodiment of the invention TSHα and/or TSHβ is linked to said receptor binding domain and is positioned carboxyl-terminal to said binding domain in said fusion protein.

In a preferred embodiment of the invention TSHα and/or TSHβ is linked to the receptor binding domain by a peptide linker; preferably a flexible peptide linker.

In a preferred embodiment of the invention said peptide linking molecule comprises at least one copy of the peptide Gly Gly Gly Gly Ser.

In a preferred embodiment of the invention said peptide linking molecule comprises 1-10 copies of the peptide Gly Gly Gly Gly Ser.

In a preferred embodiment of the invention said peptide linking molecule comprises 2, 3, 4, 5, 6, 7, 8, 9 and 10 copies of the peptide Gly Gly Gly Gly Ser. Preferably said peptide linking molecule comprises 4 or 7 copies.

In an alternative preferred embodiment of the invention said peptide linker comprises an inflexible helical region.

In an embodiment of the invention a flexible non-helical region is located at or near the amino-terminal end of the peptide linker molecule thereby allowing the orientation of TSHα and/or TSHβ located at the amino-terminal end of the peptide linker molecule in relation to said TSH receptor binding domain.

In an alternative embodiment of the invention a flexible non-helical region is located at or near the carboxyl-terminal end of the peptide linker molecule thereby allowing the orientation of TSHα and/or TSHβ located at the carboxyl-terminal end of the peptide linker molecule in relation to said TSH receptor binding domain.

In a still further embodiment of the invention a flexible non-helical region is located at or near the amino and the carboxyl-terminal ends of the peptide linker molecule linking the TSHα and/or TSHβ and TSH receptor binding domains.

In a preferred embodiment of the invention the inflexible helical region comprises at least one copy of the motif A(EAAAK)ₓA.

The length of the inflexible non-helical region is extendable by increasing the number of repeats of this A (EAAAK)ₓA motif.

In a preferred embodiment of the invention, x in the A (EAAAK)ₓA motif is less than 5 copies. Even more preferably still x is selected from 1, 2, 3, 4 or 5 copies.

In a preferred embodiment of the invention said linker consists of an inflexible alpha helical linker between said peptide binding domain and said receptor binding domain.

In a preferred embodiment of the invention said peptide linker molecule is modified to include at least one motif for the addition of at least one sugar moiety.

In a preferred embodiment of the invention said peptide linker molecule comprises at least one copy of the motif (Xaa₁ Xaa₂ Xaa₃ Xaa₄ Xaa₅) wherein said motif comprises the glycosylation motif Asn-Xaa-Ser or Asn-Xaa-Thr.

In a preferred embodiment of the invention said peptide linker comprises at least one copy of an amino acid motif selected from the group consisting of:

Asn₁-Xaa₂-Ser₃ Xaa₄ Xaa₅ wherein Xaa₂ is any amino acid except proline;
Xaa₁ Asn₂-Xaa₃-Ser₄ Xaa₅ wherein Xaa₃ is any amino acid except proline;
Xaa₁ Xaa₂ Asn₃-Xaa₄-Ser₅ wherein Xaa₄ is any amino acid except proline;
Asn₁-Xaa₂-Thr₃ Xaa₄ Xaa₅ wherein Xaa₂ is any amino acid except proline;
Xaa₁ Asn₂-Xaa₃-Thr₄ Xaa₅ wherein Xaa₃ is any amino acid except proline; and
Xaa₁ Xaa₂ Asn₃-Xaa₄-Thr₅ wherein Xaa₄ is any amino acid except proline.

Preferably said peptide linker comprises at least one copy of a motif selected from the group consisting of:

Asn₁-Xaa₂-Ser₃ Gly₄ Ser₅ wherein Xaa₂ is any amino acid except proline;
Gly₁ Asn₂-Xaa₃-Ser₄ Ser₅ wherein Xaa₃ is any amino acid except proline;
Gly₁ Gly₂ Asn₃-Xaa₄-Ser₅ wherein Xaa₄ is any amino acid except proline;
Asn₁-Xaa₂-Thr₃ Gly₄ Ser₅ wherein Xaa₂ is any amino acid except proline;
Gly₁ Asn₂-Xaa₃-Thr₄ Ser₅ wherein Xaa₃ is any amino acid except proline; and
Gly₁ Gly₂ Asn₃-Xaa₄-Thr₅ wherein Xaa₄ is any amino acid except proline.

In an alternative preferred embodiment of the invention said peptide linker comprises at least one copy of a motif selected from the group consisting of:

Asn₁-Xaa₂-Ser₃ Ser₄ Gly₅ wherein Xaa₂ is any amino acid except proline;
Ser₁ Asn₂-Xaa₃-Ser₄ Gly₅ wherein Xaa₃ is any amino acid except proline;
Ser₁ Ser₂ Asn₃-Xaa₄-Ser₅ wherein Xaa₄ is any amino acid except proline;
Asn₁-Xaa₂-Thr₃ Ser₄ Gly₅ wherein Xaa₂ is any amino acid except proline;
Ser₁ Asn₂-Xaa₃-Thr₄ Gly₅ wherein Xaa₃ is any amino acid except proline; and
Ser₁ Ser₂ Asn₃-Xaa₄-Thr₅ wherein Xaa₄ is any amino acid except proline.

In a preferred embodiment of the invention said peptide linker molecule comprises at least one copy of the motif (Xaa₁ Xaa₂ Xaa₃ Xaa₄ Xaa₅) wherein said motif comprises the glycosylation motif Asn-Xaa-Ser or Asn-Xaa-Thr and at least one copy of the motif (Gly Gly Gly Gly Ser) wherein said peptide linker is 5-50 amino acids.

In a preferred embodiment of the invention said peptide linker comprises at least one copy of the motif (Xaa₁ Xaa₂ Xaa₃ Xaa₄ Xaa₅) wherein said motif comprises the glycosylation motif Asn-Xaa-Ser or Asn-Xaa-Thr and a copy of the motif (Ser Ser Ser Ser Gly) wherein said peptide linker is 5-50 amino acids.

In a preferred embodiment of the invention said fusion polypeptide linker is modified by the addition of at least one sugar selected from the group consisting of: mannose, galactose, n-acetyl glucosamine, n-acetyl neuraminic, acid n-glycolyl neuraminic acid, n-acetyl galactosamine, fucose, glucose, rhamnose, xylose, or a combinations of sugars, for example in an oligosacharride or scaffolded system.

Suitable carbohydrate moieties include monosaccharides, oligosaccharides and polysaccharides, and include any carbohydrate moiety that is present in naturally occurring glycoproteins or in biological systems. For example, optionally protected glycosyl or glycoside derivatives, for example optionally-protected glucosyl, glucoside, galactosyl or galactoside derivatives. Glycosyl and glycoside groups include both α and β groups. Suitable carbohydrate moieties include glucose, galactose, fucose, GlcNAc, GalNAc, sialic acid, and mannose, and oligosaccharides or polysaccharides comprising at least one glucose, galactose, fucose, GlcNAc, GalNAc, sialic acid, and/or mannose residue.

Any functional groups in the carbohydrate moiety may optionally be protected using protecting groups known in the art (see for example Greene et al, “Protecting groups in organic synthesis”, 2nd Edition, Wiley, New York, 1991, the disclosure of which is hereby incorporated by reference). Suitable protecting groups for any —OH groups in the carbohydrate moiety include acetate (Ac), benzyl (Bn), silyl (for example tert-butyl dimethylsilyl (TBDMSi) and tert-butyldiphenylsilyl (TMDPSi)), acetals, ketals, and methoxymethyl (MOM). Any protecting groups may be removed before or after attachment of the carbohydrate moiety to the peptide linker.

In a preferred embodiment of the invention said sugars are unprotected.

Particularly preferred carbohydrate moieties include Glc(Ac)₄β-, Glc(Bn)₄β-, Gal(Ac)₄β-, Gal(Bn)₄β-, Glc(Ac)₄α(1,4)Glc(Ac)₃α(1,4)Glc(Ac)₄β-, β-Glc, β-Gal, -Et-β-Gal, -Et-β-Glc, Et-α-Glc, -Et-α-Man, -Et-Lac, -β-Glc(Ac)₂, -β-Glc(Ac)₃, -Et-α-Glc(Ac)₂, -Et-α-Glc(Ac)₃, -Et-α-Glc(Ac)₄, -Et-β-Glc(Ac)₂, -Et-β-Glc(Ac)₃, -Et-β-Glc(Ac)₄, -Et-α-Man(Ac)₃, -Et-α-Man(Ac)₄, -Et-β-Gal(Ac)₃, -Et-β-Gal(Ac)₄, -Et-Lac(Ac)₅, -Et-Lac(Ac)₆, -Et-Lac(Ac)₇, and their deprotected equivalents.

Preferably, any saccharide units making up the carbohydrate moiety which are derived from naturally occurring sugars will each be in the naturally occurring enantiomeric form, which may be either the D-form (e.g. D-glucose or D-galactose), or the L-form (e.g. L-rhamnose or L-fucose). Any anomeric linkages may be α- or β-linkages.

In a still further alternative embodiment of the invention said fusion protein does not comprise a peptide linking molecule and is a direct fusion of the peptide and the receptor binding domain.

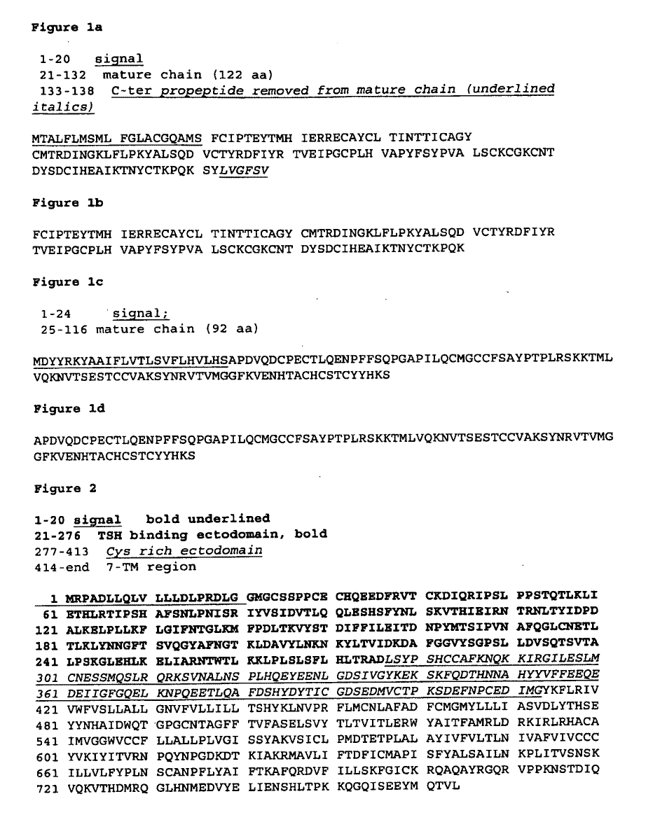

In a preferred embodiment of the invention said fusion protein comprises or consists of the amino acid as represented in FIG. 1.

In a preferred embodiment of the invention said fusion protein comprises or consists of the extracellular domain of the TSH receptor as represented in FIG. 2 with reference to Table 2.

In a preferred embodiment of the invention said fusion protein is selected from the group consisting of:
i) a protein comprising an amino acid sequence as represented in FIG. 4a, 4b or 4c;
ii) a protein comprising an amino acid sequence as represented in FIG. 4a, 4b or 4c that is modified by addition, or deletion or substitution of at least one amino acid residue and that modulates TSH receptor activity.

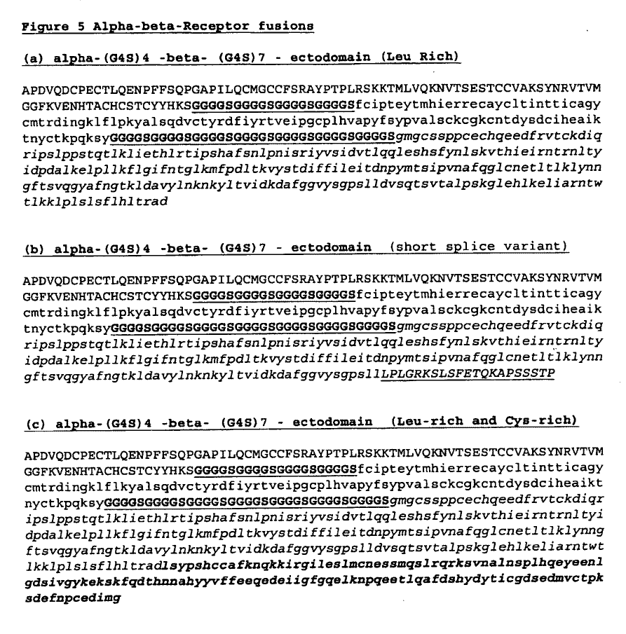

In a preferred embodiment of the invention said fusion protein is selected from the group consisting of:
i) a protein comprising an amino acid sequence as represented in FIG. 5a, 5b or 5c;
ii) a protein comprising an amino acid sequence as represented in FIG. 5a, 5b or 5c that is modified by addition, or deletion or substitution of at least one amino acid residue and that modulates TSH receptor activity.

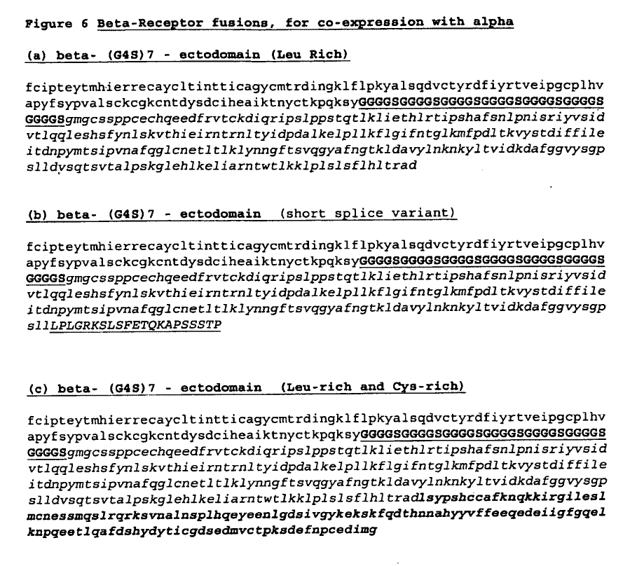

In a preferred embodiment of the invention said fusion protein is selected from the group consisting of:
i) a protein comprising an amino acid sequence as represented in FIG. 6a, 6b or 6c;
ii) a protein comprising an amino acid sequence as represented in FIG. 6a, 6b or 6c that is modified by addition, or deletion or substitution of at least one amino acid residue and that modulates TSH receptor activity.

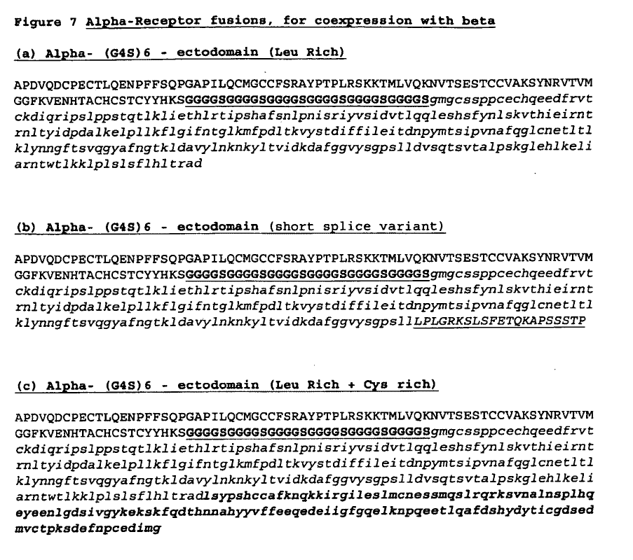

In a preferred embodiment of the invention said fusion protein is selected from the group consisting of:
i) a protein comprising an amino acid sequence as represented in FIG. 7a, 7b or 7c;
ii) a protein comprising an amino acid sequence as represented in FIG. 7a, 7b or 7c that is modified by addition, or deletion or substitution of at least one amino acid residue and that modulates TSH receptor activity.

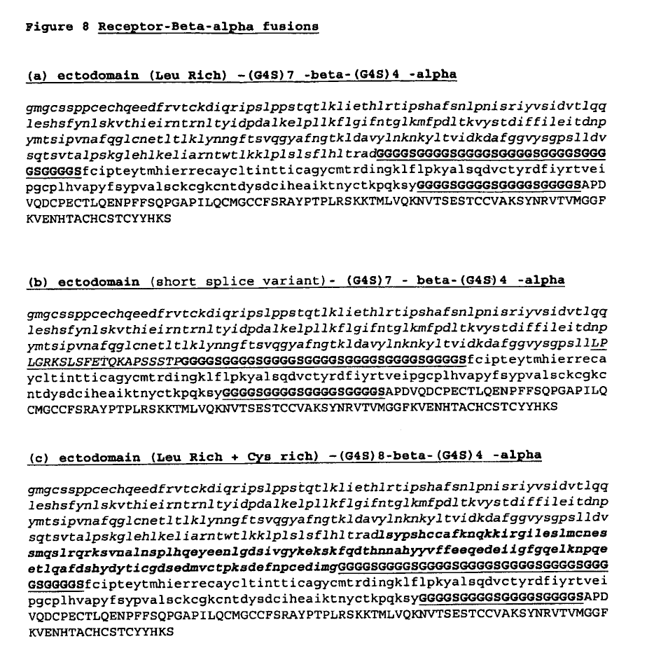

In a preferred embodiment of the invention said fusion protein is selected from the group consisting of:
i) a protein comprising an amino acid sequence as represented in FIG. 8a, 8b or 8c;
ii) a protein comprising an amino acid sequence as represented in FIG. 8a, 8b or 8c that is modified by addition, or deletion or substitution of at least one amino acid residue and that modulates TSH receptor activity.

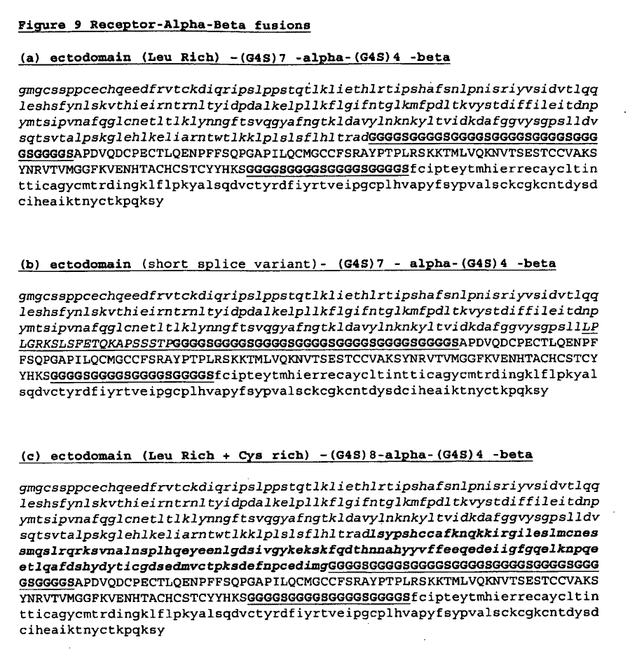

In a preferred embodiment of the invention said fusion protein is selected from the group consisting of:
i) a protein comprising an amino acid sequence as represented in FIG. 9a, 9b or 9c;
ii) a protein comprising an amino acid sequence as represented in FIG. 9a, 9b or 9c that is modified by addition, or deletion or substitution of at least one amino acid residue and that modulates TSH receptor activity.

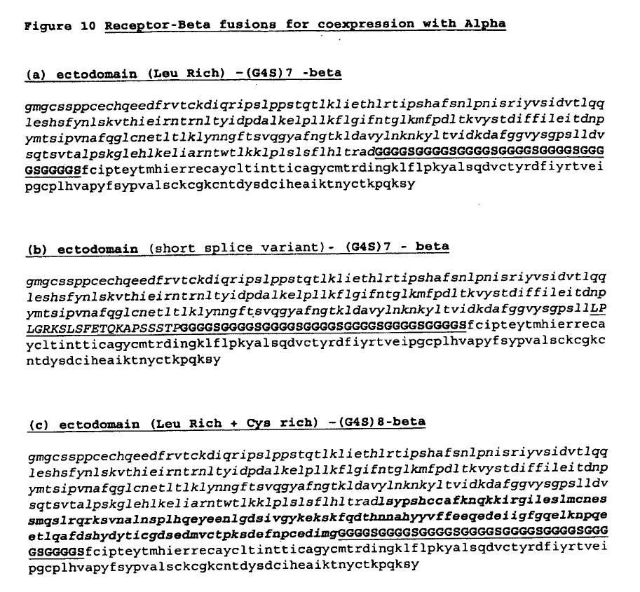

In a preferred embodiment of the invention said fusion protein is selected from the group consisting of:
i) a protein comprising an amino acid sequence as represented in FIG. 10a, 10b or 10c;
ii) a protein comprising an amino acid sequence as represented in FIG. 10a, 10b or 10c that is modified by addition, or deletion or substitution of at least one amino acid residue and that modulates TSH receptor activity.

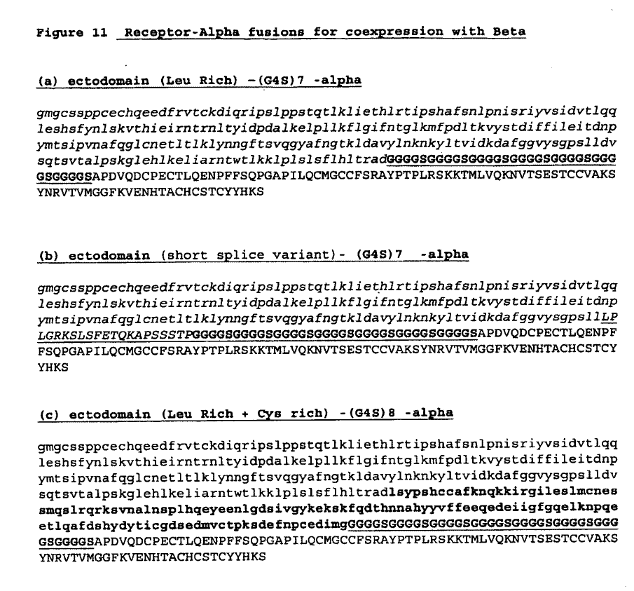

In a preferred embodiment of the invention said fusion protein is selected from the group consisting of:
i) a protein comprising an amino acid sequence as represented in FIG. 11a, 11b or 11c;
ii) a protein comprising an amino acid sequence as represented in FIG. 11a, 11b or 11c that is modified by addition, or deletion or substitution of at least one amino acid residue and that modulates TSH receptor activity.

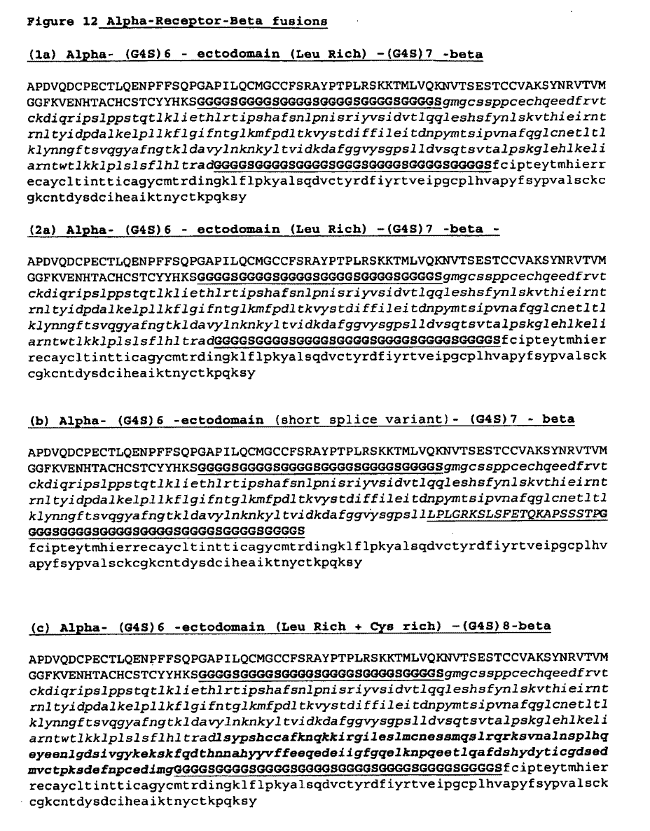

In a preferred embodiment of the invention said fusion protein is selected from the group consisting of:
i) a protein comprising an amino acid sequence as represented in FIG. 12.1a, 12.2a, 12b or 12c;
ii) a protein comprising an amino acid sequence as represented in FIG. 12a, 12b or 12c that is modified by addition, or deletion or substitution of at least one amino acid residue and that that modulates TSH receptor activity.

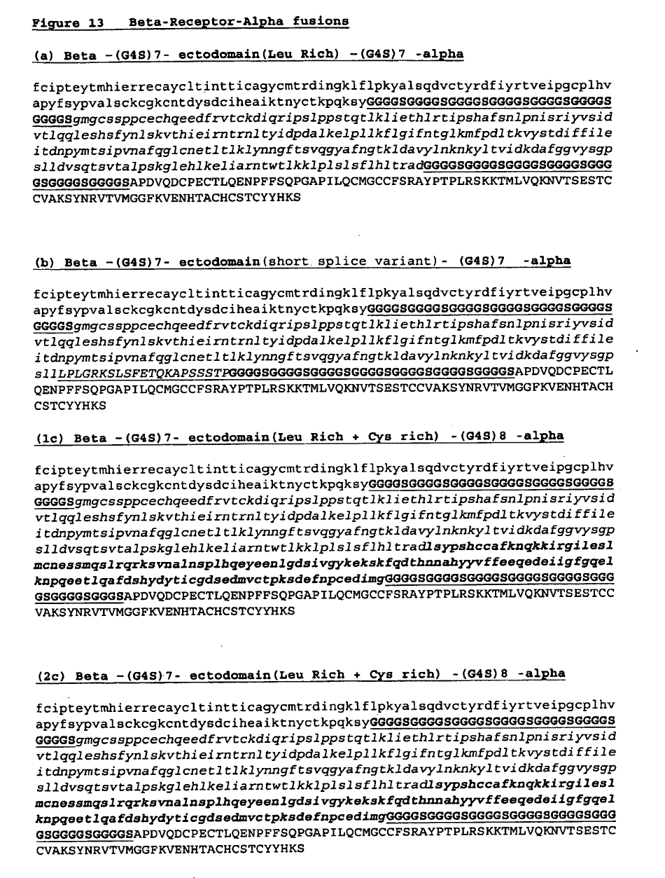

In a preferred embodiment of the invention said fusion protein is selected from the group consisting of:
i) a protein comprising an amino acid sequence as represented in FIG. 13a, 13b or 13.1c, 13.2c;
ii) a protein comprising an amino acid sequence as represented in FIG. 13a, 13b or 13c that is modified by addition, or deletion or substitution of at least one amino acid residue and that that modulates TSH receptor activity.

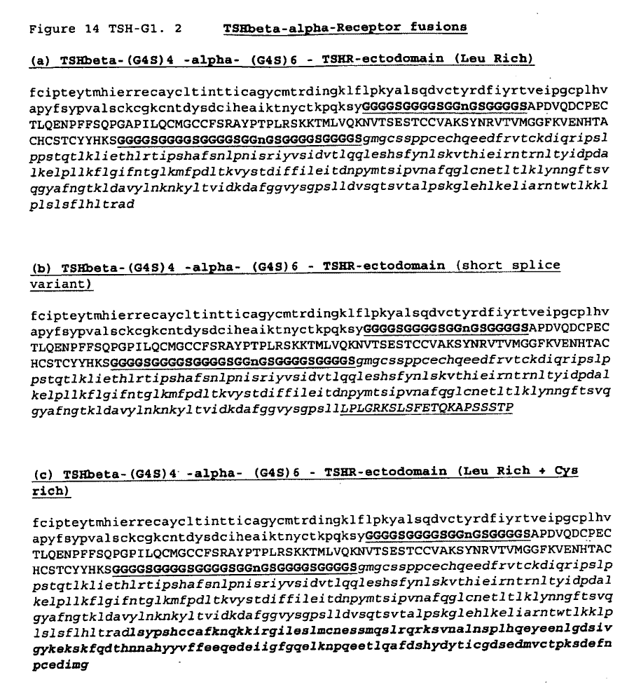

In a preferred embodiment of the invention said fusion protein is selected from the group consisting of:
i) a protein comprising an amino acid sequence as represented in FIG. 14a, 14b or 14c;
ii) a protein comprising an amino acid sequence as represented in FIG. 14a, 14b or 14c that is modified by addition, or deletion or substitution of at least one amino acid residue and that that modulates TSH receptor activity.

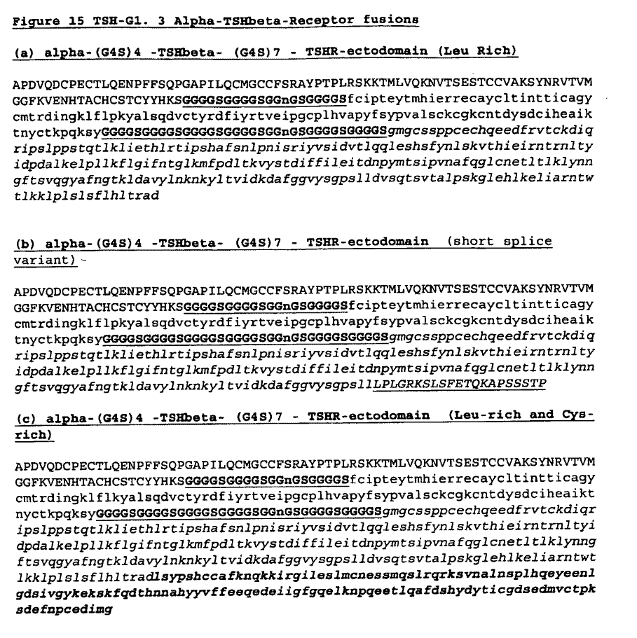

In a preferred embodiment of the invention said fusion protein is selected from the group consisting of:
i) a protein comprising an amino acid sequence as represented in FIG. 15a, 15b or 15c;
ii) a protein comprising an amino acid sequence as represented in FIG. 15a, 15b or 15c that is modified by addition, or deletion or substitution of at least one amino acid residue and that that modulates TSH receptor activity.

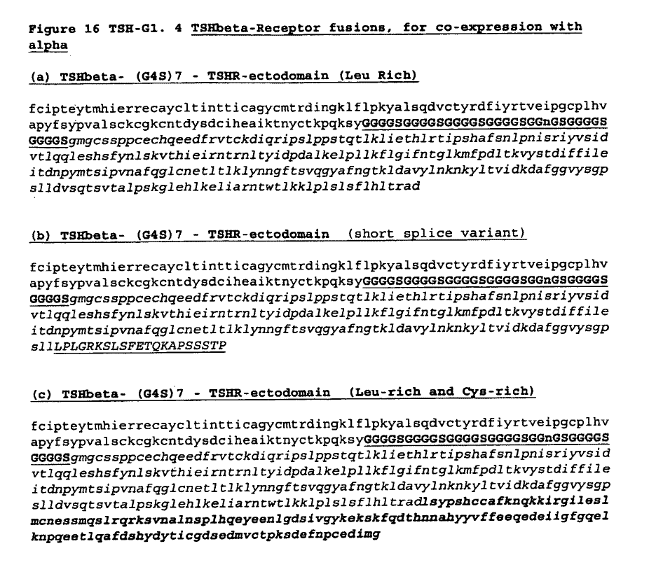

In a preferred embodiment of the invention said fusion protein is selected from the group consisting of:
i) a protein comprising an amino acid sequence as represented in FIG. 16a, 16b or 16c;
ii) a protein comprising an amino acid sequence as represented in FIG. 16a, 16b or 16c that is modified by addition, or deletion or substitution of at least one amino acid residue and that that modulates TSH receptor activity.

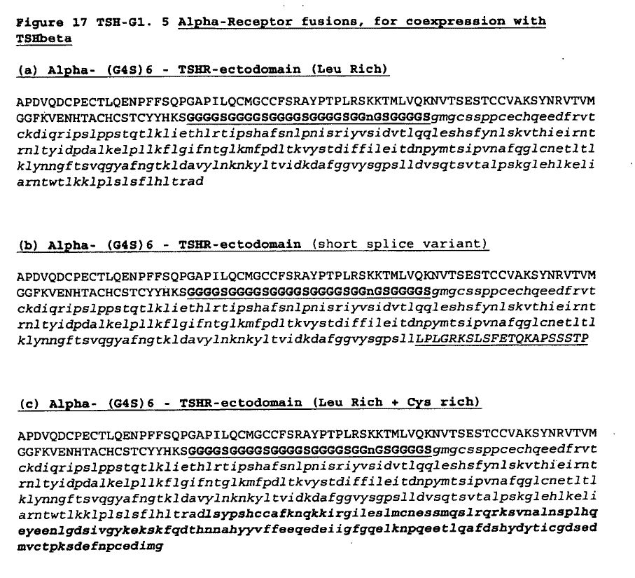

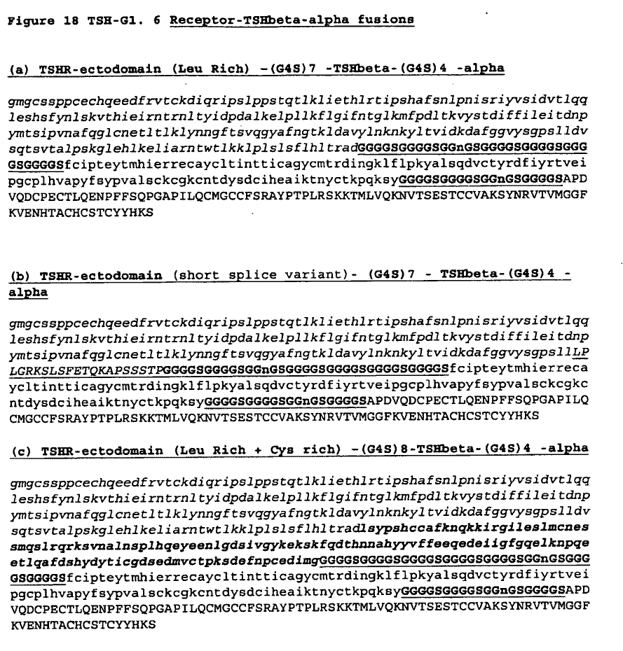

In a preferred embodiment of the invention said fusion protein is selected from the group consisting of:
i) a protein comprising an amino acid sequence as represented in FIG. 17a, 17b or 17c;
ii) a protein comprising an amino acid sequence as represented in FIG. 17a, 17b or 17c that is modified by addition, or deletion or substitution of at least one amino acid residue and that that modulates TSH receptor activity.

In a preferred embodiment of the invention said fusion protein is selected from the group consisting of:
i) a protein comprising an amino acid sequence as represented in FIG. 18a, 18b or 18c;
ii) a protein comprising an amino acid sequence as represented in FIG. 18a, 18b or 18c that is modified by addition, or deletion or substitution of at least one amino acid residue and that that modulates TSH receptor activity.

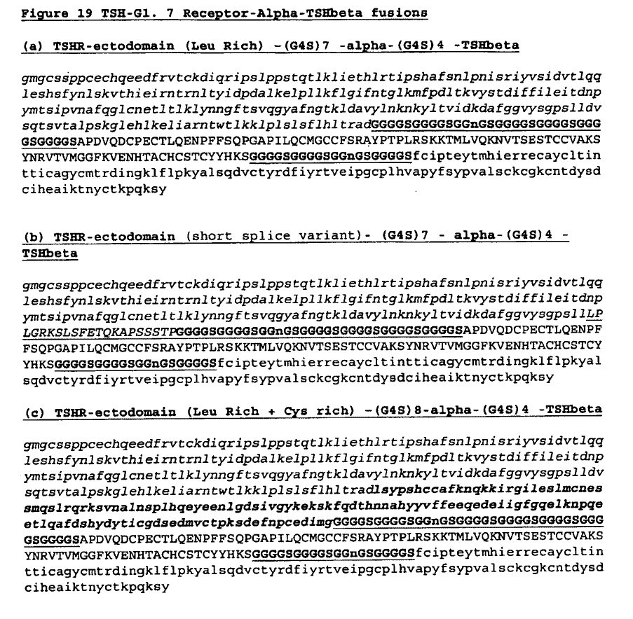

In a preferred embodiment of the invention said fusion protein is selected from the group consisting of:
i) a protein comprising an amino acid sequence as represented in FIG. 19a, 19b or 19c;
ii) a protein comprising an amino acid sequence as represented in FIG. 19a, 19b or 19c that is modified by addition, or deletion or substitution of at least one amino acid residue and that that modulates TSH receptor activity.

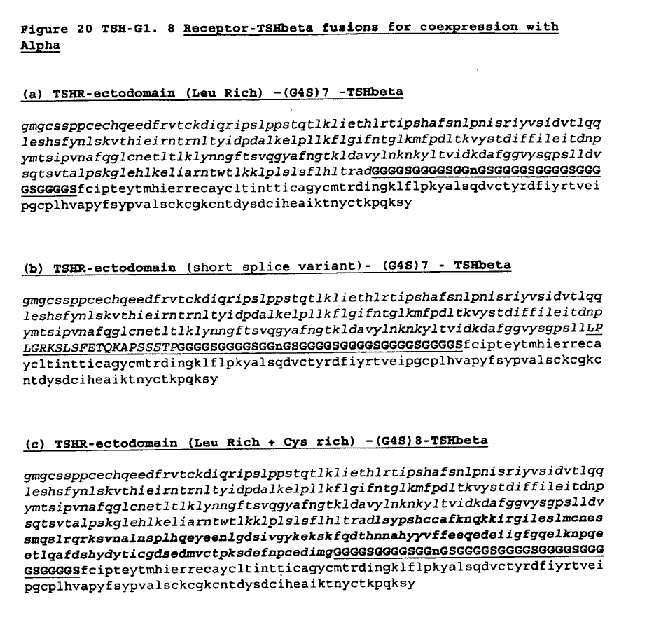

In a preferred embodiment of the invention said fusion protein is selected from the group consisting of:
i) a protein comprising an amino acid sequence as represented in FIG. 20a, 20b or 20c;
ii) a protein comprising an amino acid sequence as represented in FIG. 20a, 20b or 20c that is modified by addition, or deletion or substitution of at least one amino acid residue and that that modulates TSH receptor activity.

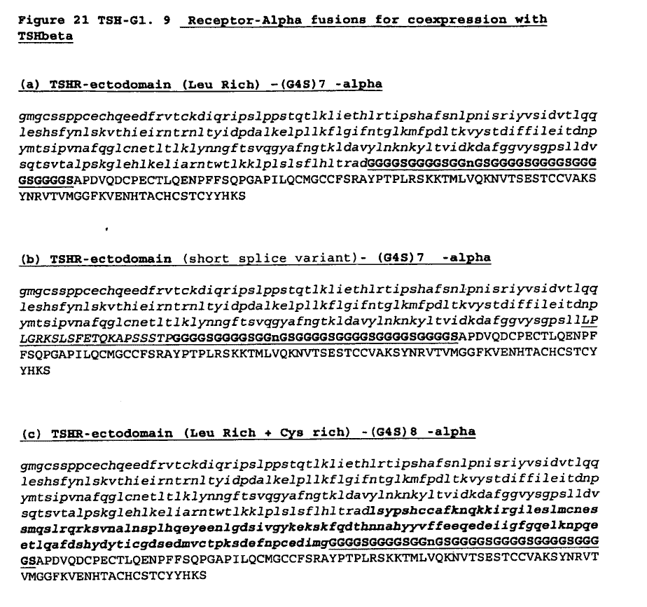

In a preferred embodiment of the invention said fusion protein is selected from the group consisting of:
i) a protein comprising an amino acid sequence as represented in FIG. 21a, 21b or 21c;
ii) a protein comprising an amino acid sequence as represented in FIG. 21a, 21b or 21c that is modified by addition, or deletion or substitution of at least one amino acid residue and that that modulates TSH receptor activity.

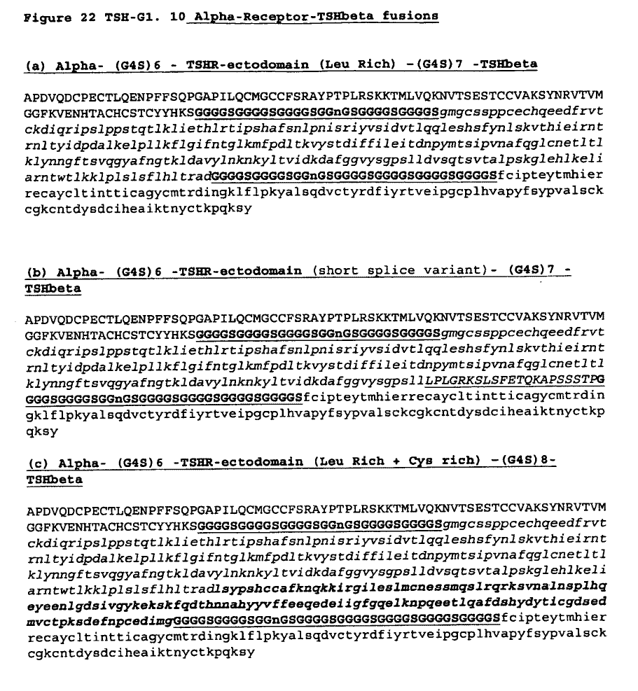

In a preferred embodiment of the invention said fusion protein is selected from the group consisting of:
i) a protein comprising an amino acid sequence as represented in FIG. 22a, 22b or 22c;
ii) a protein comprising an amino acid sequence as represented in FIG. 22a, 22b or 22c that is modified by addition, or deletion or substitution of at least one amino acid residue and that that modulates TSH receptor activity.

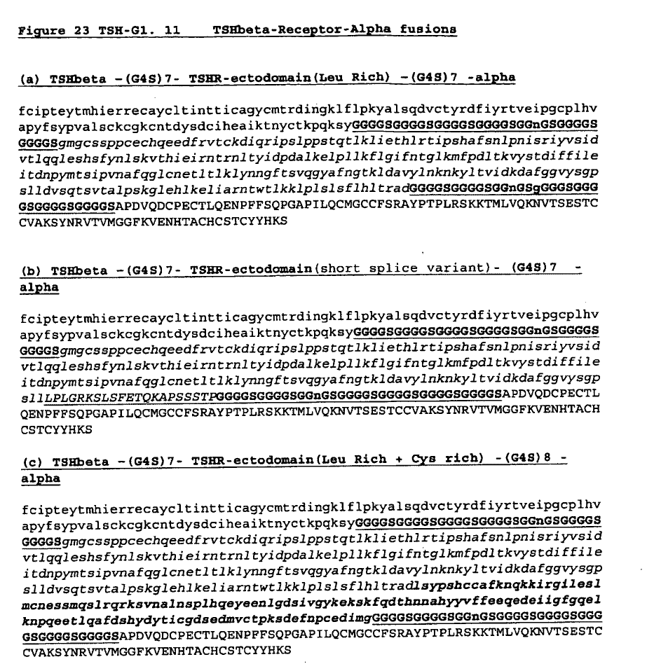

In a preferred embodiment of the invention said fusion protein is selected from the group consisting of:
i) a protein comprising an amino acid sequence as represented in FIG. 23a, 23b or 23c;
ii) a protein comprising an amino acid sequence as represented in FIG. 23a, 23b or 23c that is modified by addition, or deletion or substitution of at least one amino acid residue and that that modulates TSH receptor activity.

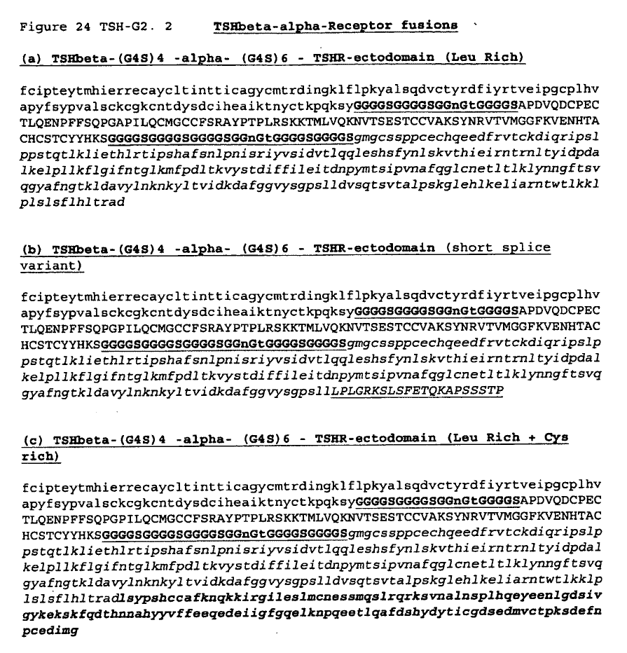

In a preferred embodiment of the invention said fusion protein is selected from the group consisting of:
i) a protein comprising an amino acid sequence as represented in FIG. 24a, 24b or 24c;
ii) a protein comprising an amino acid sequence as represented in FIG. 24a, 24b or 24c that is modified by addition, or deletion or substitution of at least one amino acid residue and that that modulates TSH receptor activity.

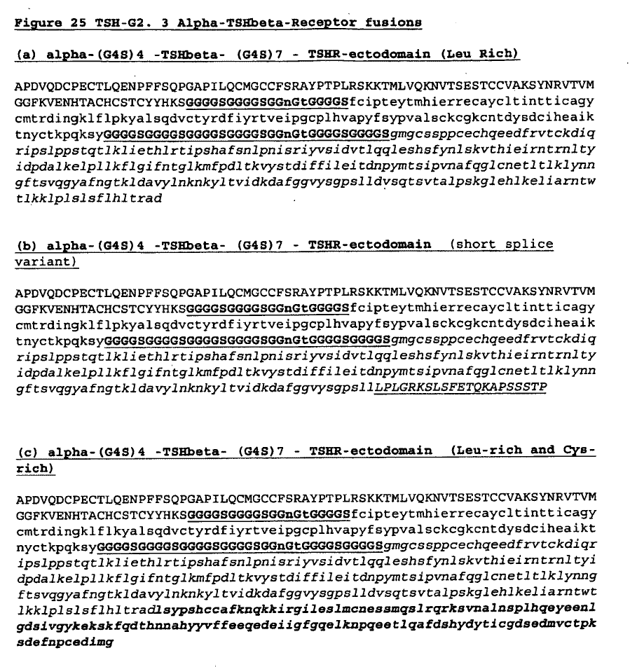

In a preferred embodiment of the invention said fusion protein is selected from the group consisting of:
i) a protein comprising an amino acid sequence as represented in FIG. 25a, 25b or 25c;
ii) a protein comprising an amino acid sequence as represented in FIG. 25a, 25b or 25c that is modified by addition, or deletion or substitution of at least one amino acid residue and that that modulates TSH receptor activity.

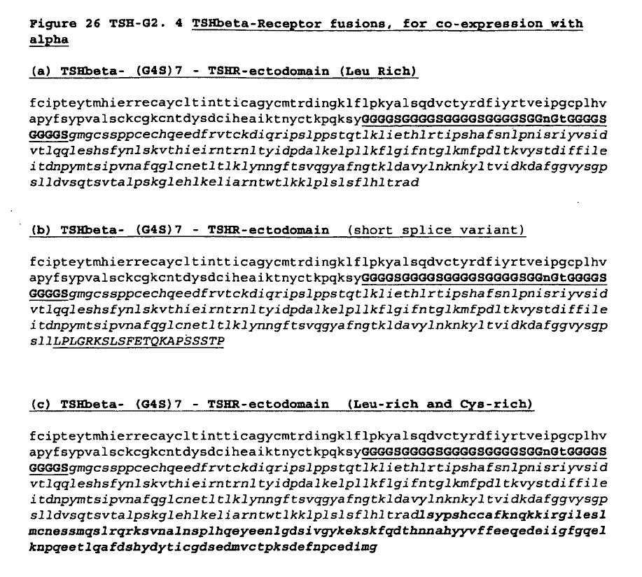

In a preferred embodiment of the invention said fusion protein is selected from the group consisting of:
i) a protein comprising an amino acid sequence as represented in FIG. 26a, 26b or 26c;
ii) a protein comprising an amino acid sequence as represented in FIG. 26a, 26b or 26c that is modified by addition, or deletion or substitution of at least one amino acid residue and that that modulates TSH receptor activity.

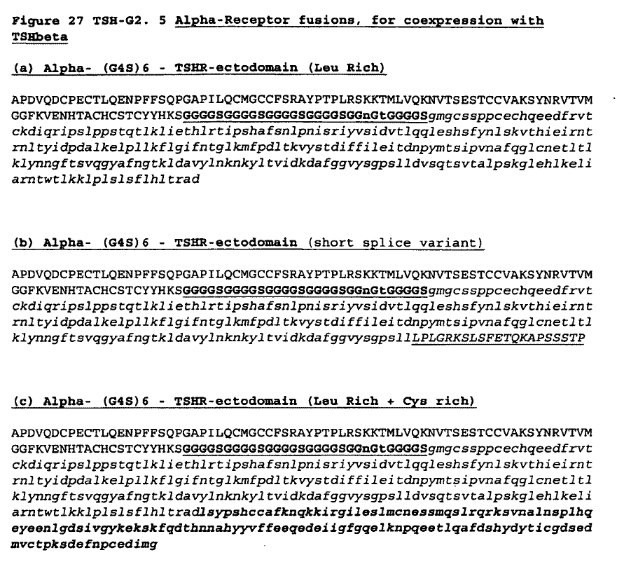

In a preferred embodiment of the invention said fusion protein is selected from the group consisting of:
i) a protein comprising an amino acid sequence as represented in FIG. 27a, 27b or 27c;
ii) a protein comprising an amino acid sequence as represented in FIG. 27a, 27b or 27c that is modified by addition, or deletion or substitution of at least one amino acid residue and that that modulates TSH receptor activity.

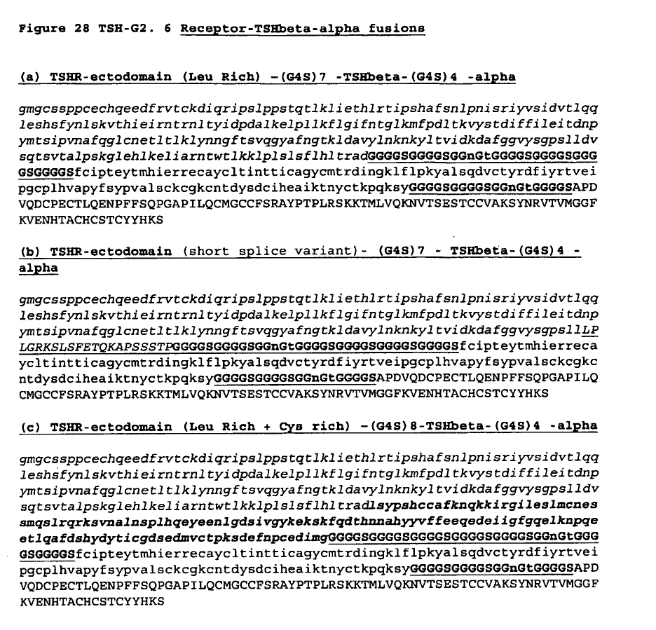

In a preferred embodiment of the invention said fusion protein is selected from the group consisting of:
i) a protein comprising an amino acid sequence as represented in FIG. 28a, 28b or 28c;
ii) a protein comprising an amino acid sequence as represented in FIG. 28a, 28b or 28c that is modified by addition, or deletion or substitution of at least one amino acid residue and that that modulates TSH receptor activity.

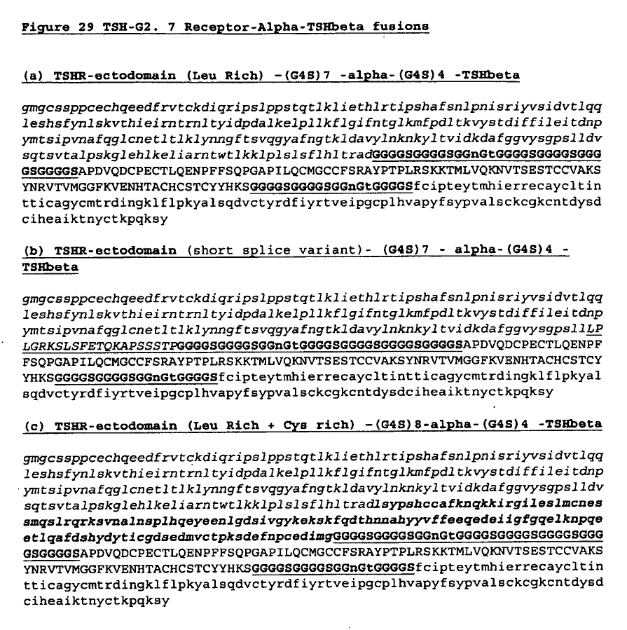

In a preferred embodiment of the invention said fusion protein is selected from the group consisting of:
i) a protein comprising an amino acid sequence as represented in FIG. 29a, 29b or 29c;
ii) a protein comprising an amino acid sequence as represented in FIG. 29a, 29b or 29c that is modified by addition, or deletion or substitution of at least one amino acid residue and that that modulates TSH receptor activity.

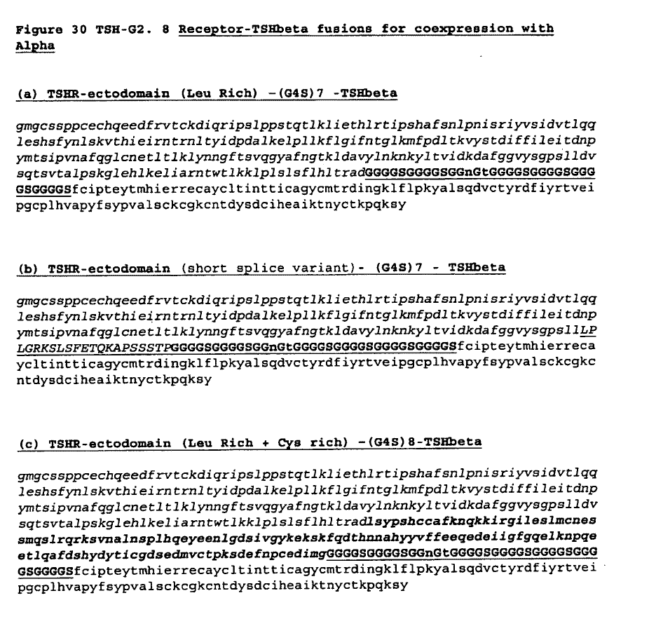

In a preferred embodiment of the invention said fusion protein is selected from the group consisting of:
i) a protein comprising an amino acid sequence as represented in FIG. 30a, 30b or 30c;
ii) a protein comprising an amino acid sequence as represented in FIG. 30a, 30b or 30c that is modified by addition, or deletion or substitution of at least one amino acid residue and that that modulates TSH receptor activity.

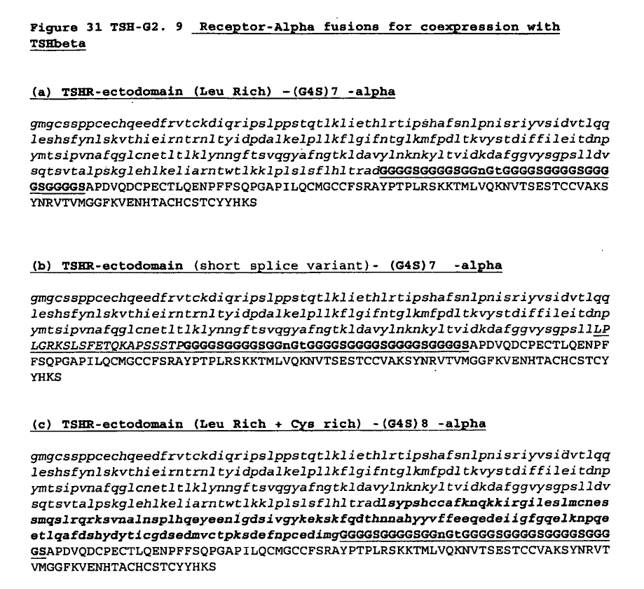

In a preferred embodiment of the invention said fusion protein is selected from the group consisting of:
i) a protein comprising an amino acid sequence as represented in FIG. 31a, 31b or 31c;
ii) a protein comprising an amino acid sequence as represented in FIG. 31a, 31b or 31c that is modified by addition, or deletion or substitution of at least one amino acid residue and that that modulates TSH receptor activity.

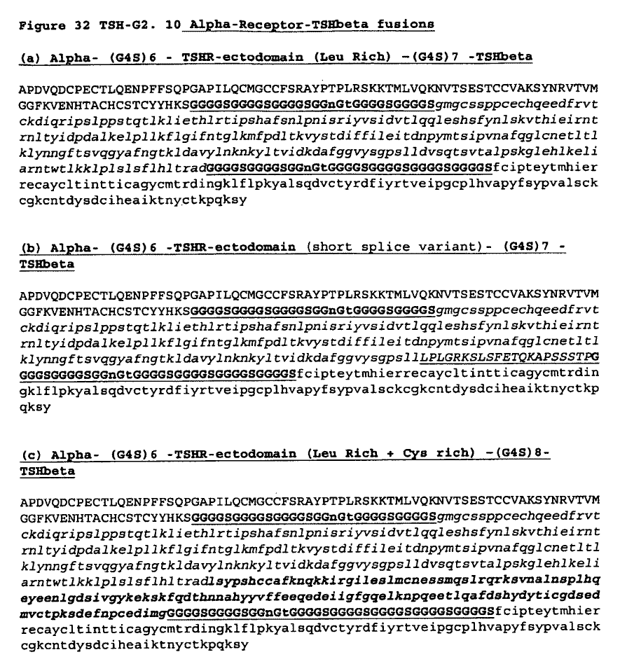

In a preferred embodiment of the invention said fusion protein is selected from the group consisting of:
i) a protein comprising an amino acid sequence as represented in FIG. 32a, 32b or 32c;
ii) a protein comprising an amino acid sequence as represented in FIG. 32a, 32b or 32c that is modified by addition, or deletion or substitution of at least one amino acid residue and that that modulates TSH receptor activity.

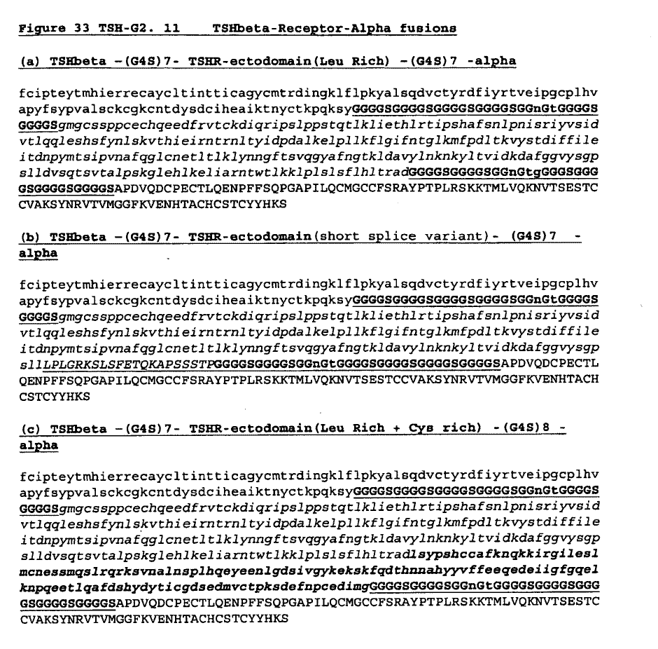

In a preferred embodiment of the invention said fusion protein is selected from the group consisting of:
i) a protein comprising an amino acid sequence as represented in FIG. 33a, 33b or 33c;
ii) a protein comprising an amino acid sequence as represented in FIG. 33a, 33b or 33c that is modified by addition, or deletion or substitution of at least one amino acid residue and that that modulates TSH receptor activity.

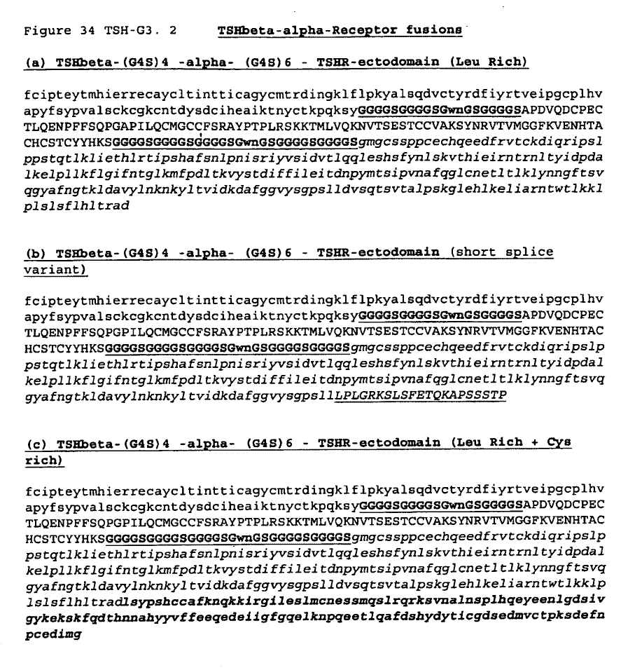

In a preferred embodiment of the invention said fusion protein is selected from the group consisting of:
i) a protein comprising an amino acid sequence as represented in FIG. 34a, 34b or 34c;
ii) a protein comprising an amino acid sequence as represented in FIG. 34a, 34b or 34c that is modified by addition, or deletion or substitution of at least one amino acid residue and that that modulates TSH receptor activity.

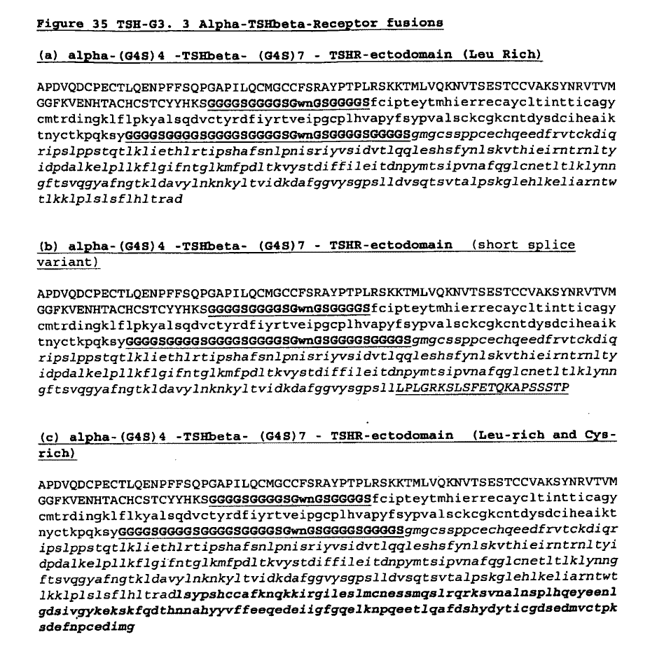

In a preferred embodiment of the invention said fusion protein is selected from the group consisting of:
i) a protein comprising an amino acid sequence as represented in FIG. 35a, 35b or 35c;
ii) a protein comprising an amino acid sequence as represented in FIG. 35a, 35b or 35c that is modified by addition, or deletion or substitution of at least one amino acid residue and that that modulates TSH receptor activity.

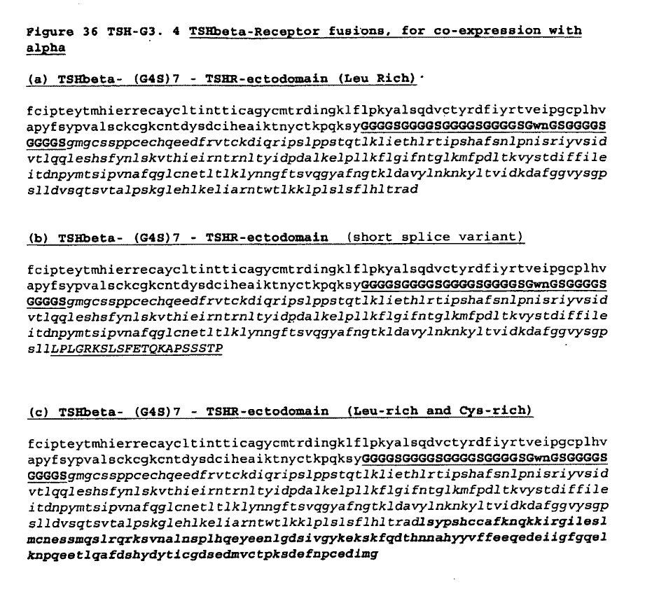

In a preferred embodiment of the invention said fusion protein is selected from the group consisting of:
i) a protein comprising an amino acid sequence as represented in FIG. 36a, 36b or 36c;
ii) a protein comprising an amino acid sequence as represented in FIG. 36a, 36b or 36c that is modified by addition, or deletion or substitution of at least one amino acid residue and that that modulates TSH receptor activity.

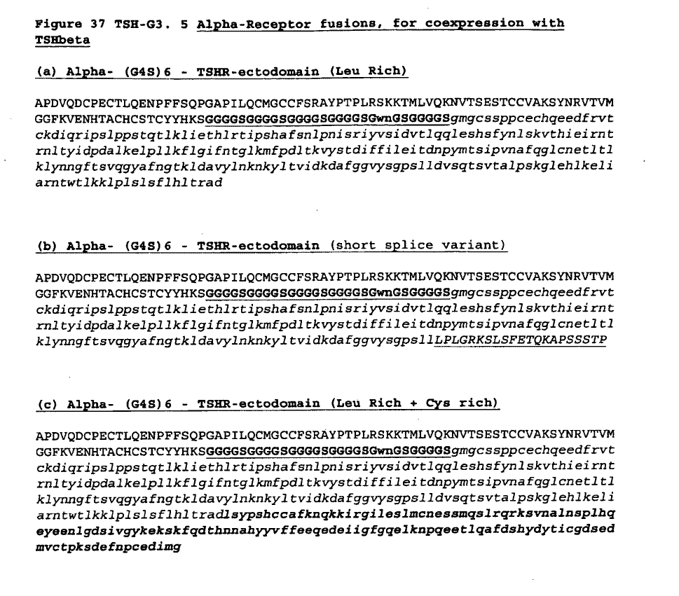

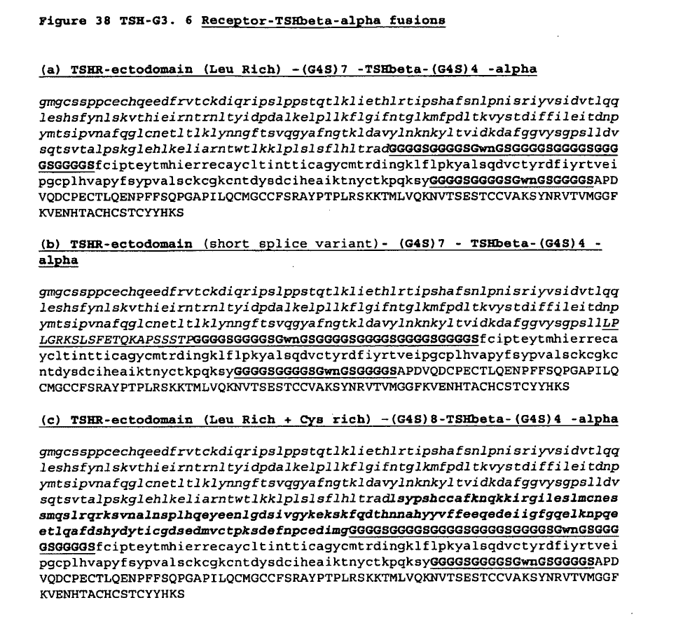

In a preferred embodiment of the invention said fusion protein is selected from the group consisting of:
i) a protein comprising an amino acid sequence as represented in FIG. 37a, 37b or 37c;
ii) a protein comprising an amino acid sequence as represented in FIG. 37a, 37b or 37c that is modified by addition, or deletion or substitution of at least one amino acid residue and that that modulates TSH receptor activity.

In a preferred embodiment of the invention said fusion protein is selected from the group consisting of:
i) a protein comprising an amino acid sequence as represented in FIG. 38a, 38b or 38c;
ii) a protein comprising an amino acid sequence as represented in FIG. 38a, 38b or 38c that is modified by addition, or deletion or substitution of at least one amino acid residue and that that modulates TSH receptor activity.

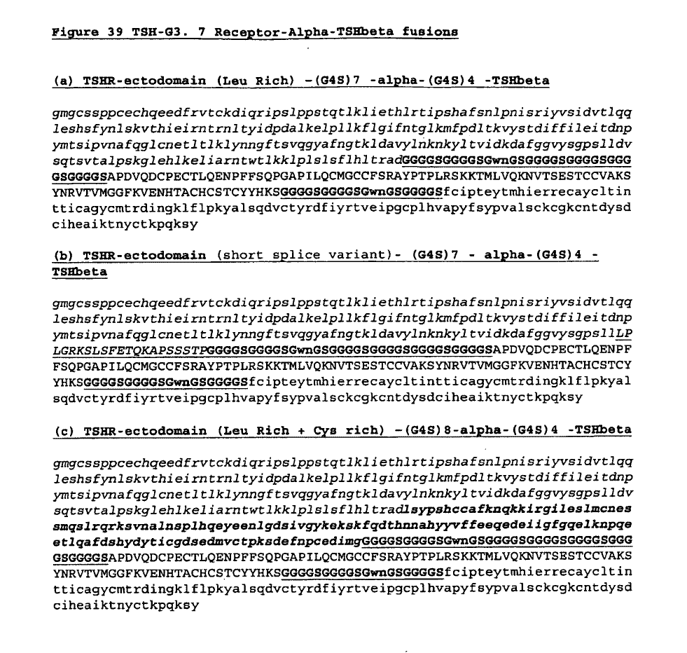

In a preferred embodiment of the invention said fusion protein is selected from the group consisting of:
i) a protein comprising an amino acid sequence as represented in FIG. 39a, 39b or 39c;
ii) a protein comprising an amino acid sequence as represented in FIG. 39a, 39b or 39c that is modified by addition, or deletion or substitution of at least one amino acid residue and that that modulates TSH receptor activity.

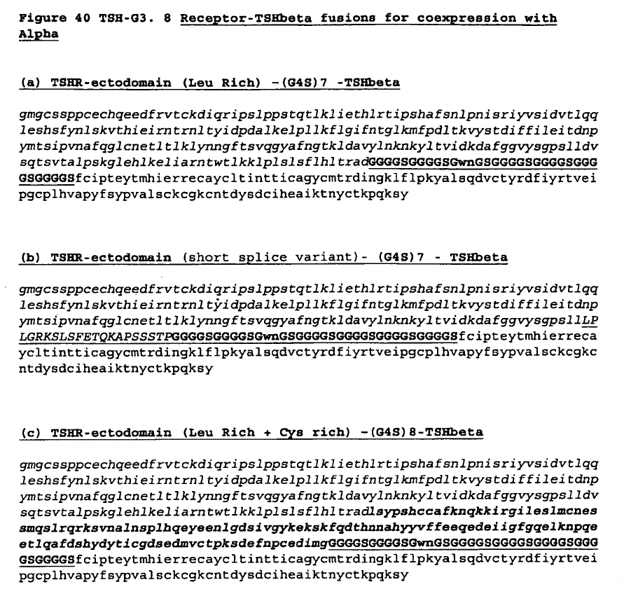

In a preferred embodiment of the invention said fusion protein is selected from the group consisting of:
i) a protein comprising an amino acid sequence as represented in FIG. 40a, 40b or 40c;
ii) a protein comprising an amino acid sequence as represented in FIG. 40a, 40b or 40c that is modified by addition, or deletion or substitution of at least one amino acid residue and that that modulates TSH receptor activity.

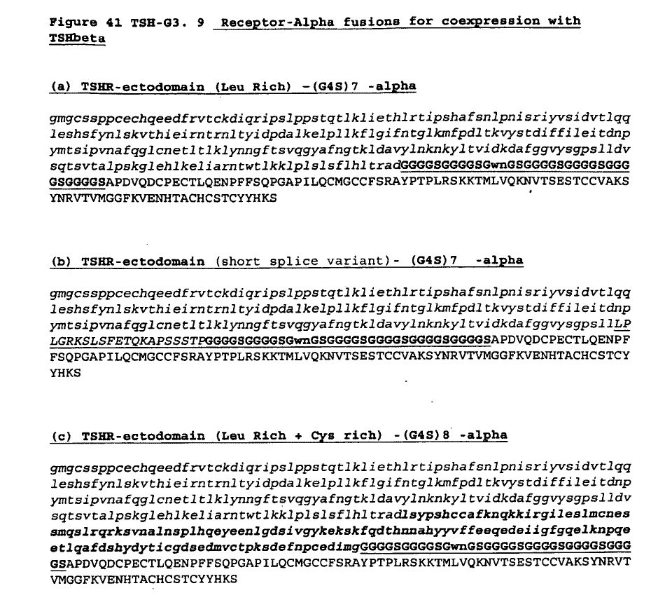

In a preferred embodiment of the invention said fusion protein is selected from the group consisting of:
i) a protein comprising an amino acid sequence as represented in FIG. 41a, 41b or 41c;
ii) a protein comprising an amino acid sequence as represented in FIG. 41a, 41b or 41c that is modified by addition, or deletion or substitution of at least one amino acid residue and that that modulates TSH receptor activity.

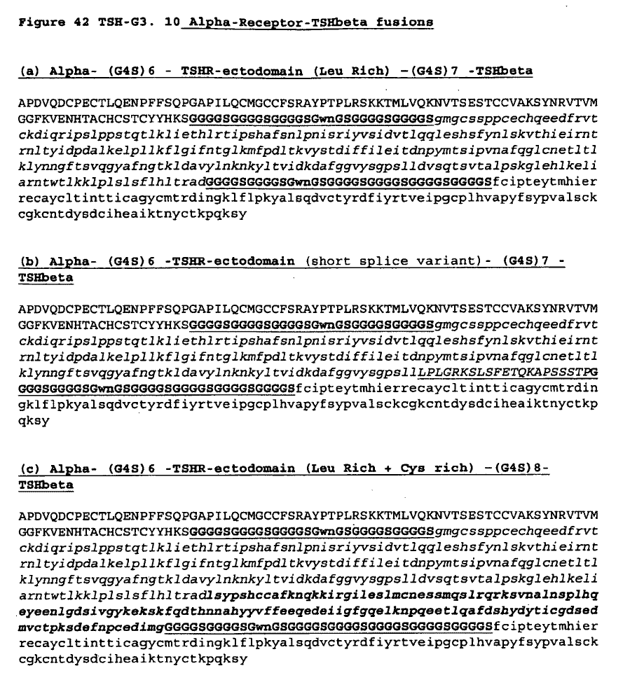

In a preferred embodiment of the invention said fusion protein is selected from the group consisting of:
i) a protein comprising an amino acid sequence as represented in FIG. 42a, 42b or 42c;
ii) a protein comprising an amino acid sequence as represented in FIG. 42a, 42b or 42c that is modified by addition, or deletion or substitution of at least one amino acid residue and that that modulates TSH receptor activity.

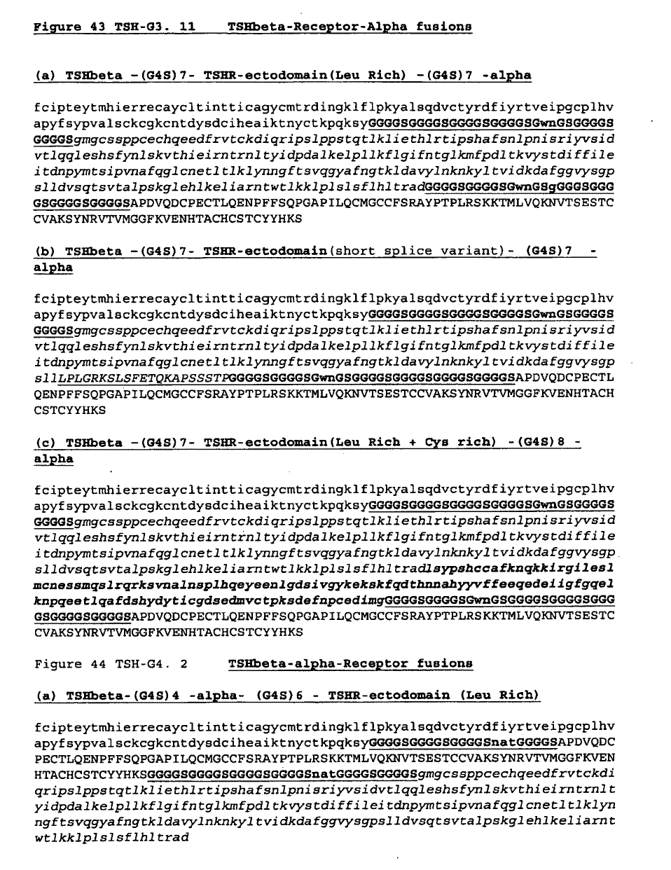

In a preferred embodiment of the invention said fusion protein is selected from the group consisting of:
i) a protein comprising an amino acid sequence as represented in FIG. 43a, 43b or 43c;
ii) a protein comprising an amino acid sequence as represented in FIG. 43a, 43b or 43c that is modified by addition, or deletion or substitution of at least one amino acid residue and that that modulates TSH receptor activity.

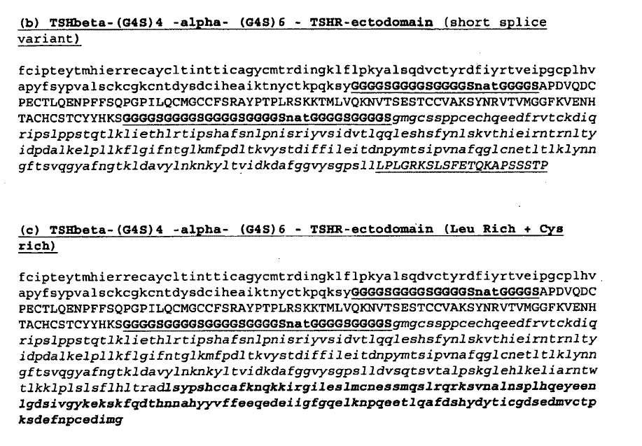

In a preferred embodiment of the invention said fusion protein is selected from the group consisting of:
i) a protein comprising an amino acid sequence as represented in FIG. 44a, 44b or 44c;
ii) a protein comprising an amino acid sequence as represented in FIG. 44a, 44b or 44c that is modified by addition, or deletion or substitution of at least one amino acid residue and that that modulates TSH receptor activity.

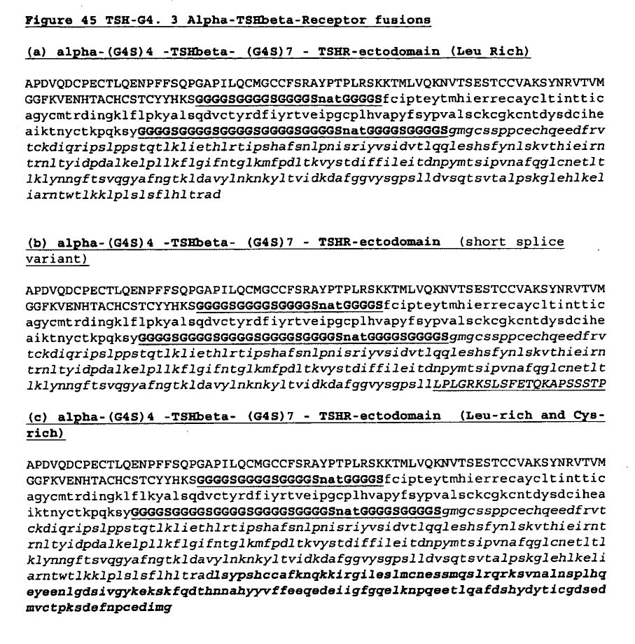

In a preferred embodiment of the invention said fusion protein is selected from the group consisting of:
i) a protein comprising an amino acid sequence as represented in FIG. 45a, 45b or 45c;
ii) a protein comprising an amino acid sequence as represented in FIG. 45a, 45b or 45c that is modified by addition, or deletion or substitution of at least one amino acid residue and that that modulates TSH receptor activity.

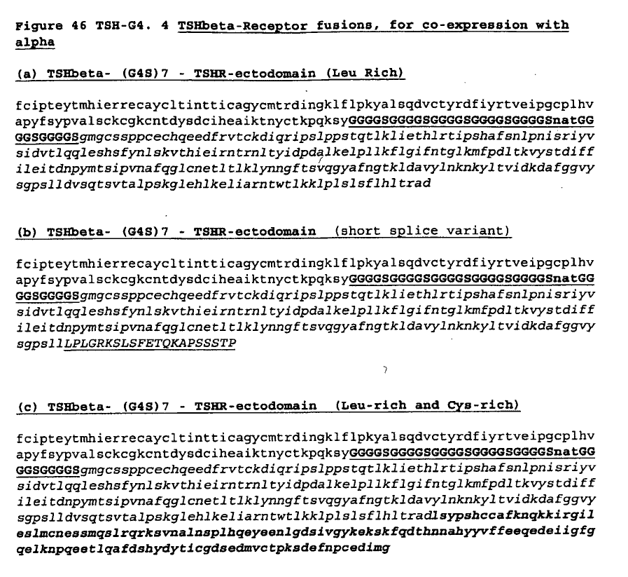

In a preferred embodiment of the invention said fusion protein is selected from the group consisting of:
i) a protein comprising an amino acid sequence as represented in FIG. 46a, 46b or 46c;
ii) a protein comprising an amino acid sequence as represented in FIG. 46a, 46b or 46c that is modified by addition, or deletion or substitution of at least one amino acid residue and that that modulates TSH receptor activity.

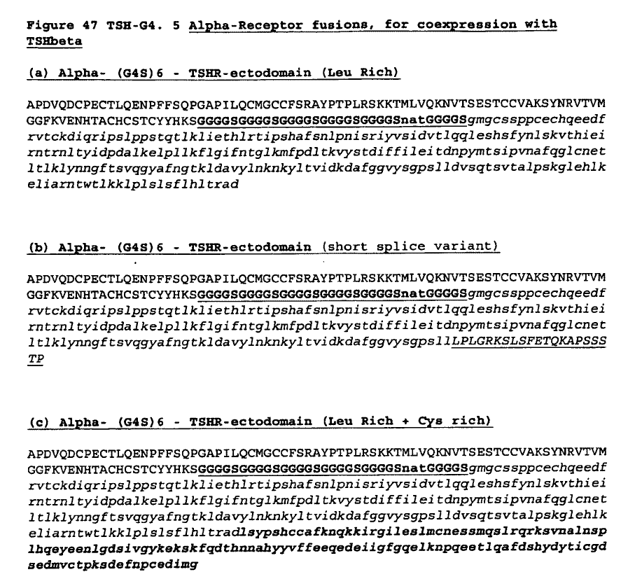

In a preferred embodiment of the invention said fusion protein is selected from the group consisting of:
i) a protein comprising an amino acid sequence as represented in FIG. 47a, 47b or 47c;
ii) a protein comprising an amino acid sequence as represented in FIG. 47a, 47b or 47c that is modified by addition, or deletion or substitution of at least one amino acid residue and that that modulates TSH receptor activity.

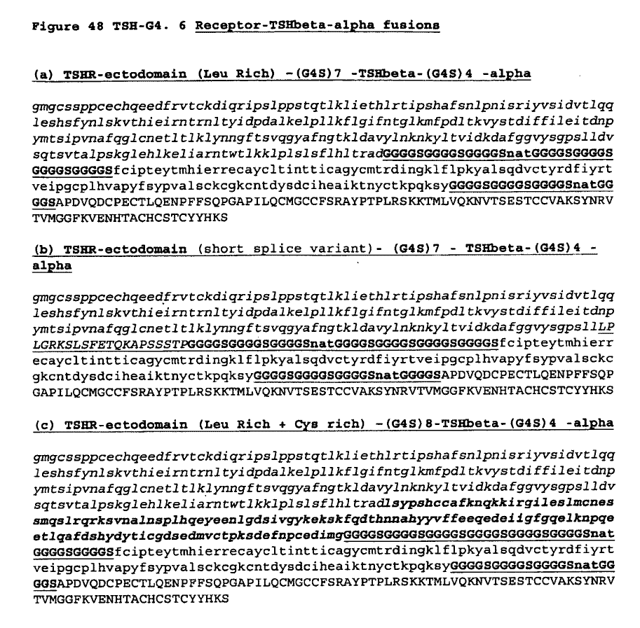

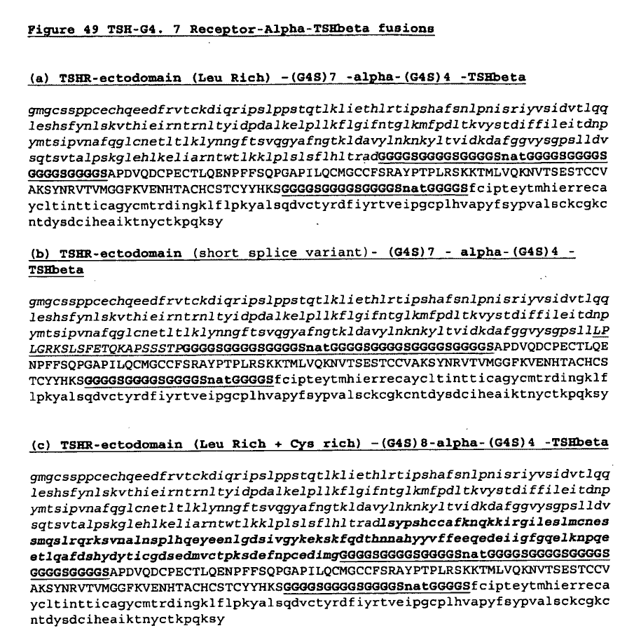

In a preferred embodiment of the invention said fusion protein is selected from the group consisting of:
i) a protein comprising an amino acid sequence as represented in FIG. 48a, 48b or 48c;
ii) a protein comprising an amino acid sequence as represented in FIG. 48a, 48b or 48c that is modified by addition, or deletion or substitution of at least one amino acid residue and that that modulates TSH receptor activity.

In a preferred embodiment of the invention said fusion protein is selected from the group consisting of:
i) a protein comprising an amino acid sequence as represented in FIG. 49a, 49b or 49c;
ii) a protein comprising an amino acid sequence as represented in FIG. 49a, 49b or 49c that is modified by addition, or deletion or substitution of at least one amino acid residue and that that modulates TSH receptor activity.

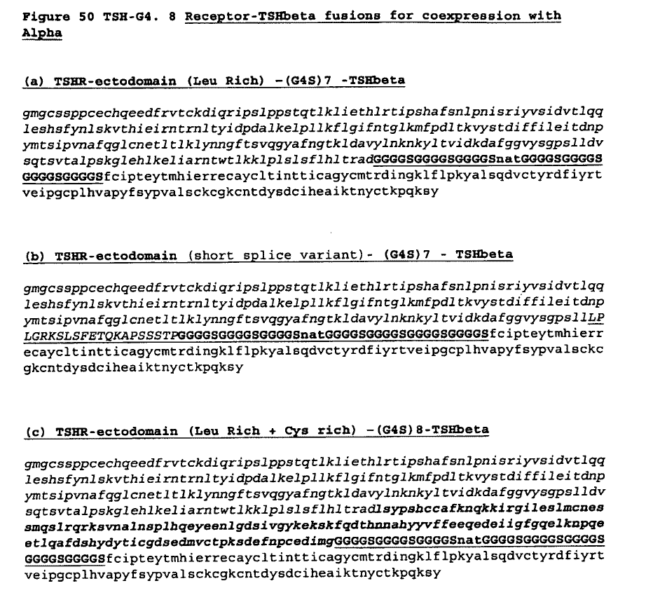

In a preferred embodiment of the invention said fusion protein is selected from the group consisting of:
i) a protein comprising an amino acid sequence as represented in FIG. 50a, 50b or 50c;
ii) a protein comprising an amino acid sequence as represented in FIG. 50a, 50b or 50c that is modified by addition, or deletion or substitution of at least one amino acid residue and that that modulates TSH receptor activity.

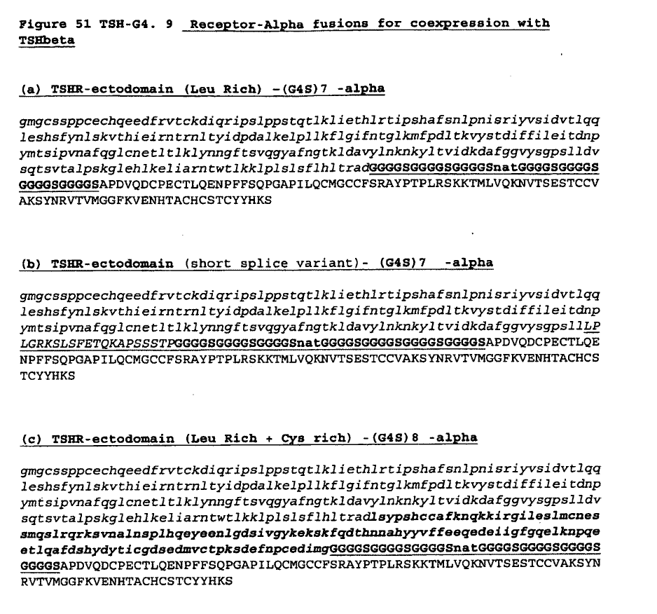

In a preferred embodiment of the invention said fusion protein is selected from the group consisting of:
i) a protein comprising an amino acid sequence as represented in FIG. 51a, 51b or 51c;
ii) a protein comprising an amino acid sequence as represented in FIG. 51a, 51b or 51c that is modified by addition, or deletion or substitution of at least one amino acid residue, and that that modulates TSH receptor activity.

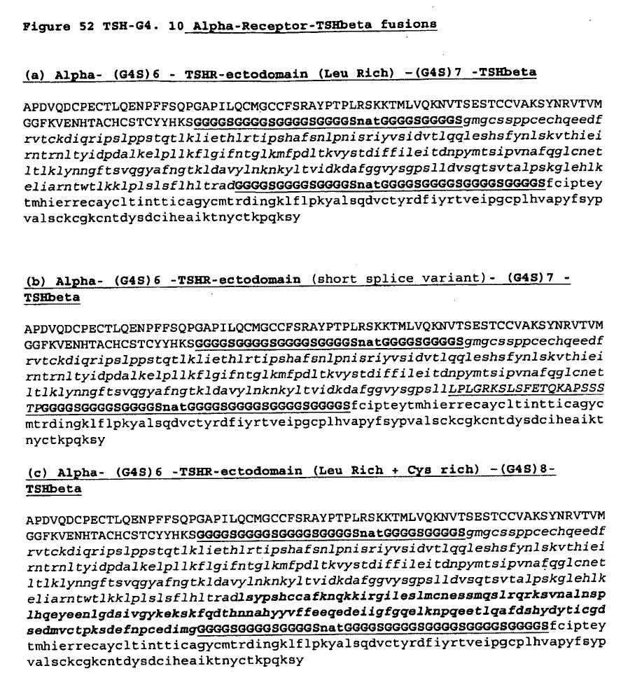

In a preferred embodiment of the invention said fusion protein is selected from the group consisting of:
i) a protein comprising an amino acid sequence as represented in FIG. 52a, 52b or 52c;
ii) a protein comprising an amino acid sequence as represented in FIG. 52a, 52b or 52c that is modified by addition, or deletion or substitution of at least one amino acid residue and that that modulates TSH receptor activity.

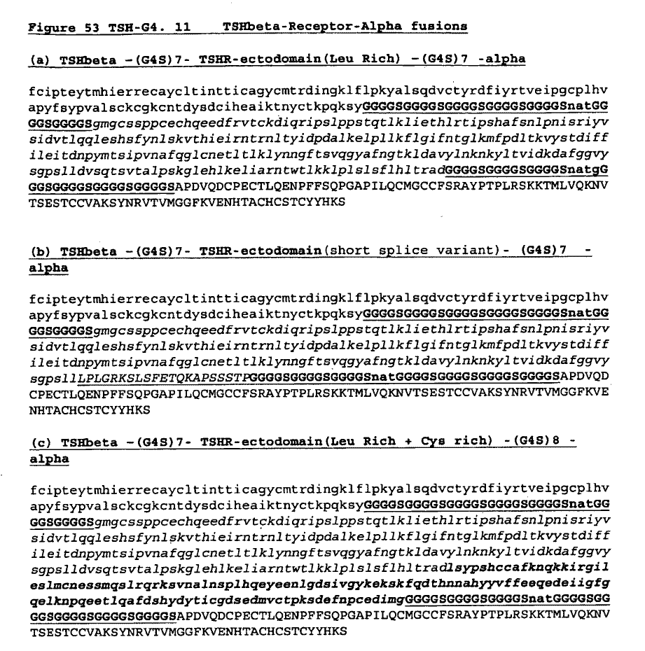

In a preferred embodiment of the invention said fusion protein is selected from the group consisting of:
i) a protein comprising an amino acid sequence as represented in FIG. 53a, 53b or 53c;
ii) a protein comprising an amino acid sequence as represented in FIG. 53a, 53b or 53c that is modified by addition, or deletion or substitution of at least one amino acid residue and that that modulates TSH receptor activity.

In a preferred embodiment of the invention said fusion protein comprises of TSHα and TSHβ linked in tandem; preferably said fusion protein consists of TSHα and TSHβ.

In a preferred embodiment of the invention said fusion protein comprises first and second TSHα and TSHβ polypeptides.

In a preferred embodiment of the invention said first and second TSHα and TSH polypeptides are linked in tandem wherein said fusion protein comprises:
i) TSHβ-TSHα-TSHβ-TSHα; or
ii) TSHα-TSHβ-TSHβ-TSHα; or
iii) TSHβ-TSHα-TSHα-TSHβ; or
iv) TSHα-TSHβ-TSHα-TSHβ.

In a preferred embodiment of the invention said fusion protein is selected from the group consisting of:
i) a protein comprising an amino acid sequence as represented in FIG. 54;
ii) a protein comprising an amino acid sequence as represented in FIG. 54 that is modified by addition, or deletion or substitution of at least one amino acid residue and that that modulates TSH receptor activity.

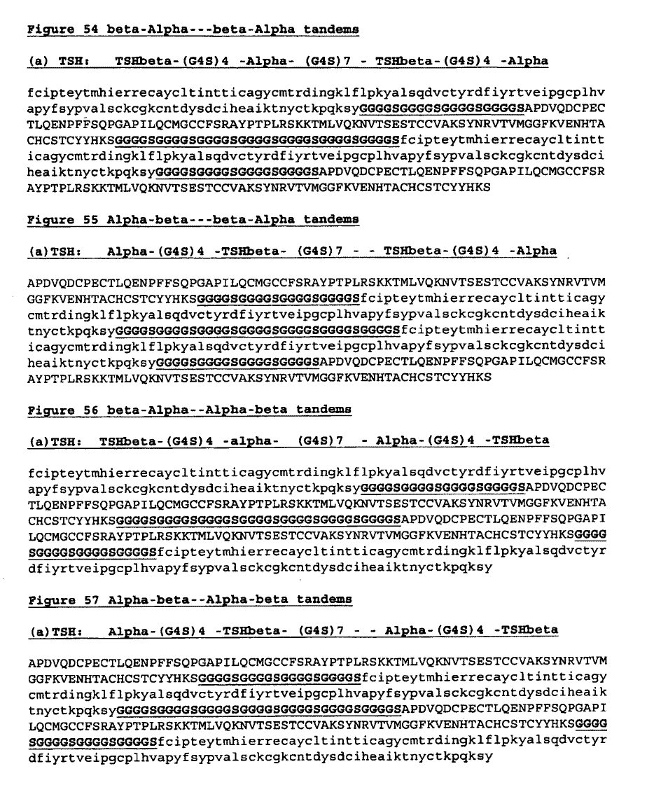

In a preferred embodiment of the invention said fusion protein is selected from the group consisting of:
i) a protein comprising an amino acid sequence as represented in FIG. 55;
ii) a protein comprising an amino acid sequence as represented in FIG. 55 that is modified by addition, or deletion or substitution of at least one amino acid residue and that that modulates TSH receptor activity.

In a preferred embodiment of the invention said fusion protein is selected from the group consisting of:
i) a protein comprising an amino acid sequence as represented in FIG. 56;
ii) a protein comprising an amino acid sequence as represented in FIG. 56 that is modified by addition, or deletion or substitution of at least one amino acid residue and that that modulates TSH receptor activity.

In a preferred embodiment of the invention said fusion protein is selected from the group consisting of:
i) a protein comprising an amino acid sequence as represented in FIG. 57;
ii) a protein comprising an amino acid sequence as represented in FIG. 57 that is modified by addition, or deletion or substitution of at least one amino acid residue and that that modulates TSH receptor activity.

In a preferred embodiment of the invention said fusion protein is selected from the group consisting of:
i) a protein comprising an amino acid sequence as represented in FIG. 58;
ii) a protein comprising an amino acid sequence as represented in FIG. 58 that is modified by addition, or deletion or substitution of at least one amino acid residue and that that modulates TSH receptor activity.

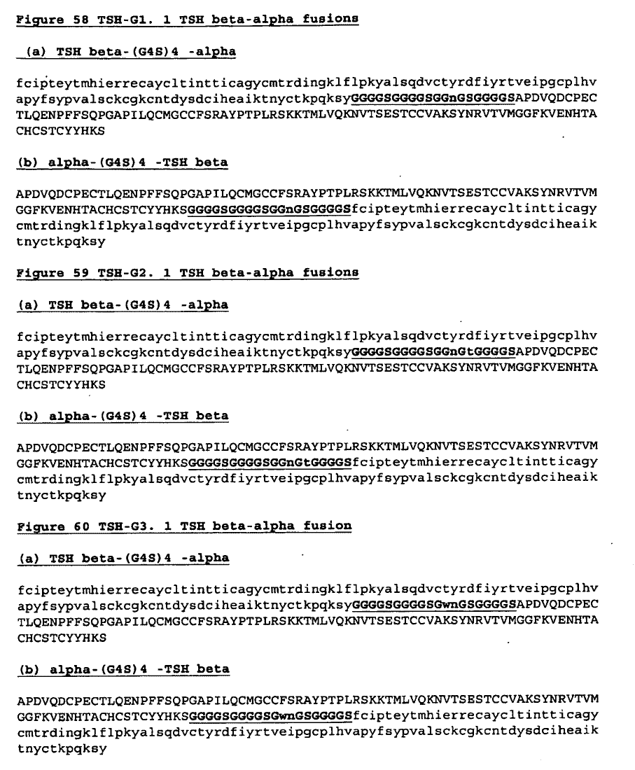

In a preferred embodiment of the invention said fusion protein is selected from the group consisting of:
i) a protein comprising an amino acid sequence as represented in FIG. 59;
ii) a protein comprising an amino acid sequence as represented in FIG. 59 that is modified by addition, or deletion or substitution of at least one amino acid residue and that that modulates TSH receptor activity.

In a preferred embodiment of the invention said fusion protein is selected from the group consisting of:
i) a protein comprising an amino acid sequence as represented in FIG. 60;
ii) a protein comprising an amino acid sequence as represented in FIG. 60 that is modified by addition, or deletion or substitution of at least one amino acid residue and that that modulates TSH receptor activity.

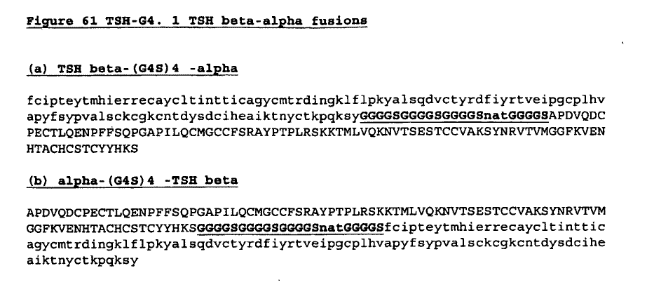

In a preferred embodiment of the invention said fusion protein is selected from the group consisting of:
i) a protein comprising an amino acid sequence as represented in FIG. 61;
ii) a protein comprising an amino acid sequence as represented in FIG. 61 that is modified by addition, or deletion or substitution of at least one amino acid residue and that that modulates TSH receptor activity.

The term “modulates” encompasses both activation and inhibition of receptor, for example TSH receptor activation. The disclosed TSH fusion proteins have utility in the treatment of conditions in which TSH receptor activation is desirable. For example, in the pretreatment of patients who are to receive radioactive iodine in the treatment of thyroid cancer and multinodular goiter or thyrotoxicosis. In these clinical conditions TSH is often suppressed and as TSH stimulates iodine uptake into the thyroid there is poor radioactive iodine uptake. Furthermore, TSH fusion proteins herein disclosed will increase radioactive iodine uptake with a greater effect of the radioactivity on the thyroid. TSH fusion proteins can be used to treat secondary hypothyroidism that is hypothyroid patients who have pituitary failure. TSH has anabolic effects on bone turnover and therefore provides a treatment for osteoporosis.

According to a further aspect of the invention there is provided a method to treat a subject suffering from cancer comprising administering an effective amount of a TSH fusion protein according to the invention.

According to a further aspect of the invention there is provided a method to treat a subject suffering from multinodular goiter comprising administering an effective amount of a TSH fusion protein according to the invention.

According to a further aspect of the invention there is provided a method to treat a subject suffering from thyrotoxicosis comprising administering an effective amount of a TSH fusion protein according to the invention.

According to a further aspect of the invention there is provided a method to treat a subject suffering from secondary hypothyroidism comprising administering an effective amount of a TSH fusion protein according to the invention.

According to a further aspect of the invention there is provided a method to treat a subject suffering from osteoporosis comprising administering an effective amount of a TSH fusion protein according to the invention.

According to an aspect of the invention there is provided a nucleic acid molecule that encodes a fusion protein according to the invention.

According to a further aspect of the invention there is provided a vector comprising a nucleic acid molecule according to the invention.

In a preferred embodiment of the invention said vector is an expression vector adapted to express the nucleic acid molecule according to the invention.

A vector including nucleic acid (s) according to the invention need not include a promoter or other regulatory sequence, particularly if the vector is to be used to introduce the nucleic acid into cells for recombination into the genome for stable transfection. Preferably the nucleic acid in the vector is operably linked to an appropriate promoter or other regulatory elements for transcription in a host cell. The vector may be a bi-functional expression vector which functions in multiple hosts. By “promoter” is meant a nucleotide sequence upstream from the transcriptional initiation site and which contains all the regulatory regions required for transcription. Suitable promoters include constitutive, tissue-specific, inducible, developmental or other promoters for expression in eukaryotic or prokaryotic cells. “Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is a constitutive, an inducible or regulatable promoter.

According to a further aspect of the invention there is provided a cell transfected or transformed with a nucleic acid molecule or vector according to the invention.

Preferably said cell is a eukaryotic cell. Alternatively said cell is a prokaryotic cell.

In a preferred embodiment of the invention said cell is selected from the group consisting of; a fungal cell (e.g. Pichia spp, Saccharomyces spp, Neurospora spp); insect cell (e.g. Spodoptera spp); a mammalian cell (e.g. COS cell, CHO cell); a plant cell.

According to a further aspect of the invention there is provided a pharmaceutical composition comprising a polypeptide according to the invention including an excipient or carrier.

In a preferred embodiment of the invention said pharmaceutical composition is combined with a further therapeutic agent.

When administered the pharmaceutical composition of the present invention is administered in pharmaceutically acceptable preparations. Such preparations may routinely contain pharmaceutically acceptable concentrations of salt, buffering agents, preservatives, compatible carriers, and optionally other therapeutic agents.

The pharmaceutical compositions of the invention can be administered by any conventional route, including injection. The administration and application may, for example, be oral, intravenous, intraperitoneal, intramuscular, intracavity, intra-articuar, subcutaneous, topical (eyes), dermal (e.g a cream lipid soluble insert into skin or mucus membrane), transdermal, or intranasal.

Pharmaceutical compositions of the invention are administered in effective amounts. An “effective amount” is that amount of pharmaceuticals/compositions that alone, or together with further doses or synergistic drugs, produces the desired response. This may involve only slowing the progression of the disease temporarily, although more preferably, it involves halting the progression of the disease permanently. This can be monitored by routine methods or can be monitored according to diagnostic methods.

The doses of the pharmaceuticals compositions administered to a subject can be chosen in accordance with different parameters, in particular in accordance with the mode of administration used and the state of the subject (i.e. age, sex). When administered, the pharmaceutical compositions of the invention are applied in pharmaceutically-acceptable amounts and in pharmaceutically-acceptable compositions. When used in medicine salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical compositions may be combined, if desired, with a pharmaceutically-acceptable carrier. The term “pharmaceutically-acceptable carrier” as used herein means one or more compatible solid or liquid fillers, diluents or encapsulating substances that are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction that would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation that is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to a further aspect of the invention there is provided a method to treat a disease or condition in a subject comprising administering an effective amount of a fusion protein according to the invention.

According to an aspect of the invention there is provided a method to treat a subject suffering from a cancer comprising administering an effective amount of a TSH fusion protein according to the invention.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following Tables and figures:

Table 1 illustrates the domains in full length TSH α and the full length amino acid sequence;

Table 2 illustrates the domains in full length TSH receptor and the full length amino acid sequence;

Table 3 illustrates the domains in full length TSH β and the full length amino acid sequence;

FIG. 1a is the amino acid sequence of unprocessed human TSH β; FIG. 1b is the amino acid sequence of processed human TSH β; FIG. 1c is unprocessed human TSH α; FIG. 1d is processed human TSH α;

FIG. 2 is the amino acid sequence of human TSH receptor;

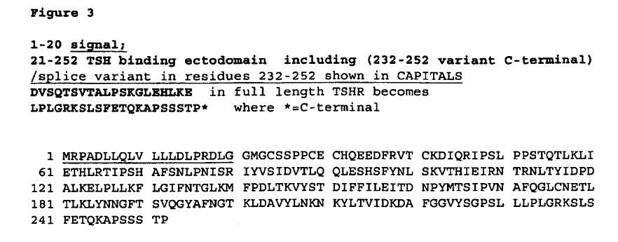

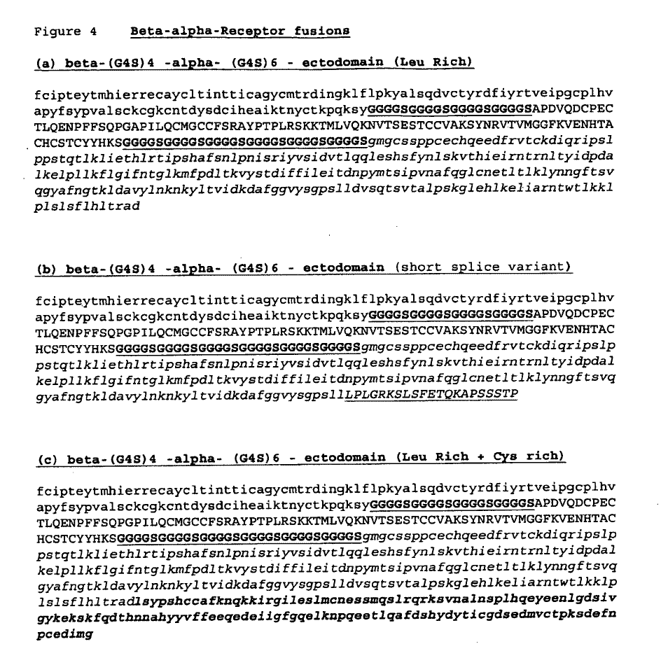

FIG. 3 is a spliced variant of human TSH receptor;

FIG. 4a-c is the amino acid sequences of human TSH β-α-TSH receptor fusion proteins;

FIG. 5a-c is the amino acid sequences of human TSH α-β-TSH receptor fusion proteins;

FIG. 6a-c is the amino acid sequences of human TSH β-TSH receptor fusion proteins;

FIG. 7a-c is the amino acid sequences of human TSH α-TSH receptor fusion proteins;

FIG. 8a-c is the amino acid sequences of human TSH receptor-TSHβ-α fusion proteins;

FIG. 9a-c is the amino acid sequences of human TSH receptor-TSHα-β fusion proteins;

FIG. 10a-c is the amino acid sequences of human TSH receptor-TSHβ fusion proteins;

FIG. 11a-c is the amino acid sequences of human TSH receptor-TSHα fusion proteins;

FIG. 12a-c is the amino acid sequence of human TSHα-TSH receptor-TSH β fusion proteins;

FIG. 13a-c is the amino acid sequence of human TSHβ-TSH-receptor-TSH α fusion proteins;

FIG. 14-FIG. 53 is the amino acid sequence of TSHα and/or TSH β fusion proteins linked via a peptide linker that includes a glycosylation motif;

FIG. 54-58 is the amino acid sequence of TSHα and TSH β tandem fusion polypeptides;

FIG. 59-61 is the amino acid sequence of TSHα and TSH β tandem fusion polypeptides linked via a peptide linker that includes a glycosylation motif;

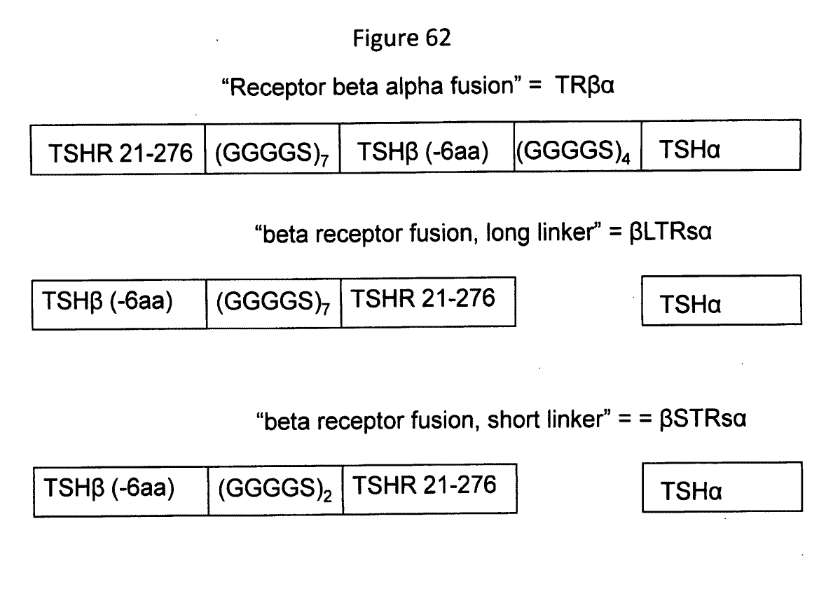

FIG. 62 shows summary and names of three constructs cloned, expressed and tested in the TSH bioassay;

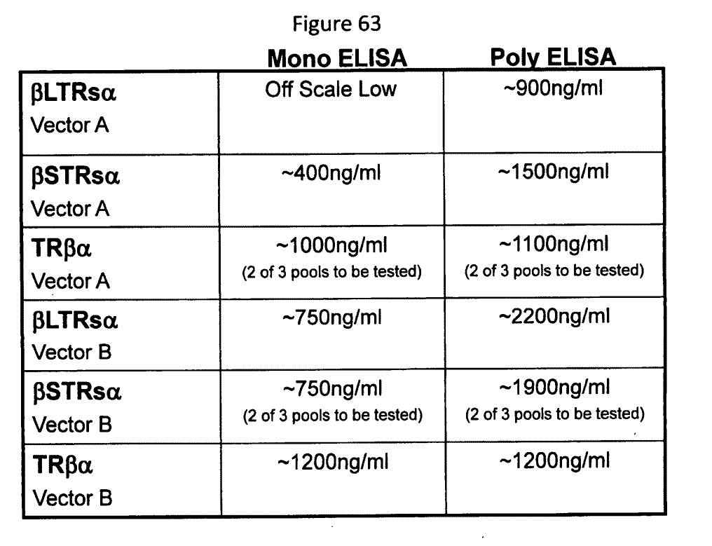

FIG. 63 shows summary data for screening pools of medium from cells expressing TSH constructs. By ELISA all constructs are expressed at a level of between 0.5 to 2 ug/ml;

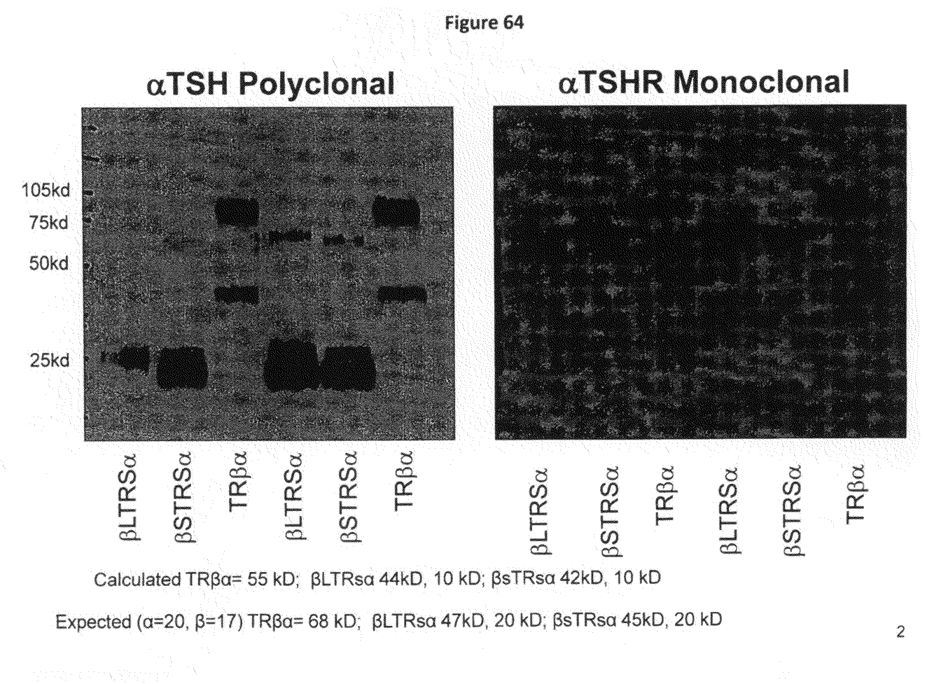

FIG. 64: Western blot of media from cells expressing TSH constructs. Left hand panel is western blot to TSH and right hand panel to TSH receptor. All the constructs are expressed according to western blot with bands at around the predicted size. All constructs demonstrate that they possess both TSH and the TSH receptor;

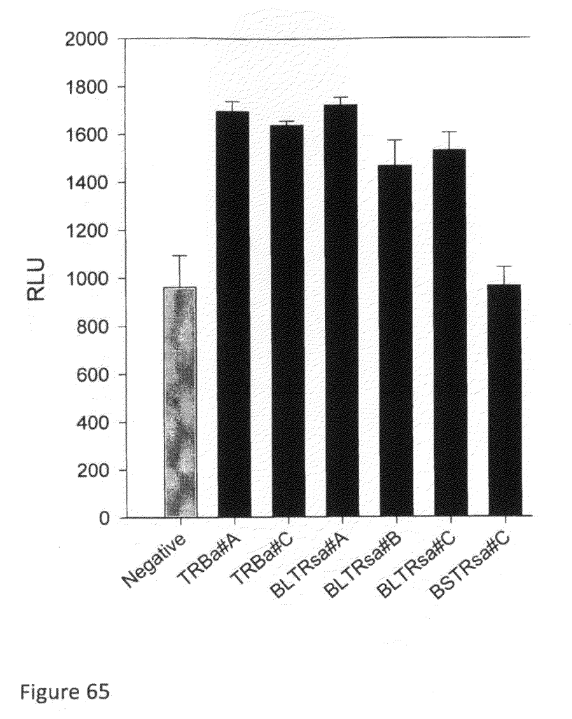

FIG. 65 show initial screen in the bioassay. All constructs show positive bioreactivity compared to negative control except for BSTRsa; and

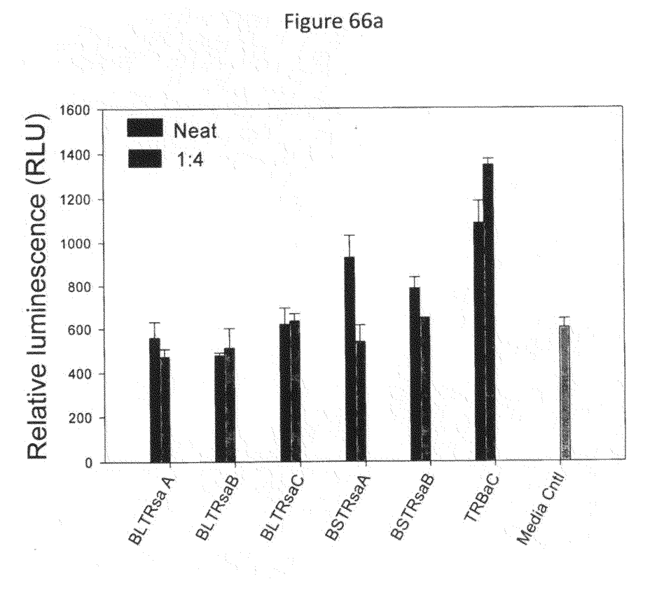

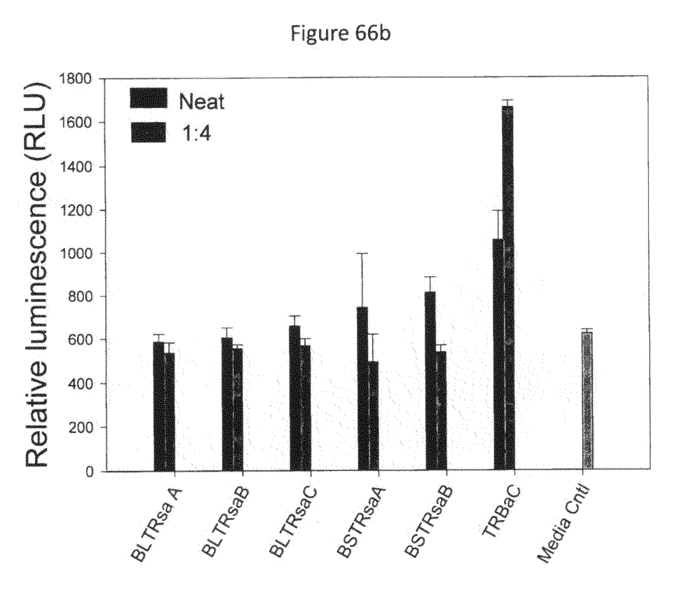

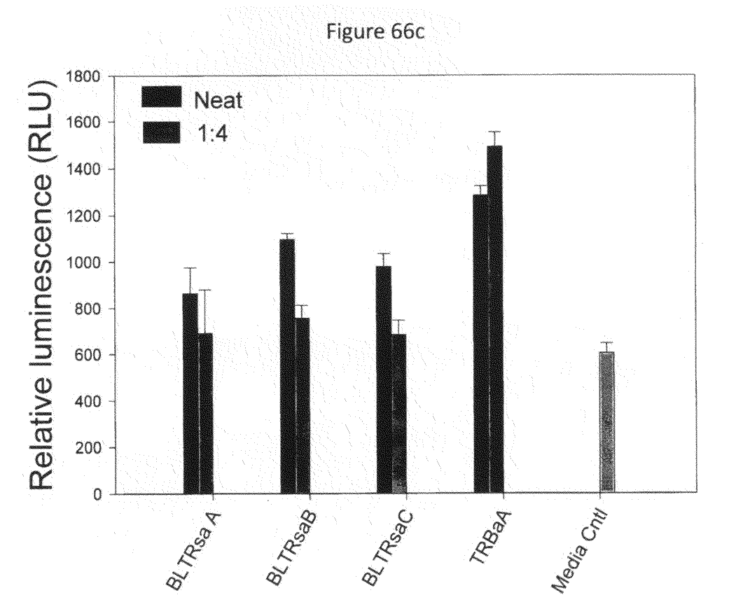

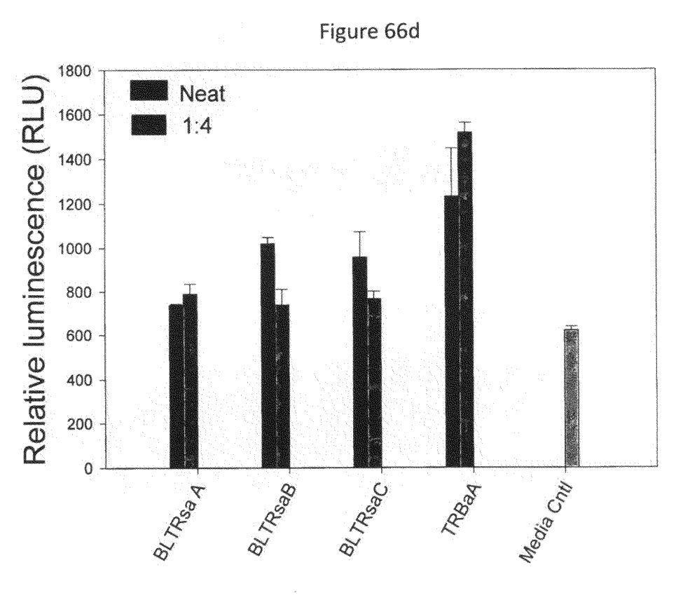

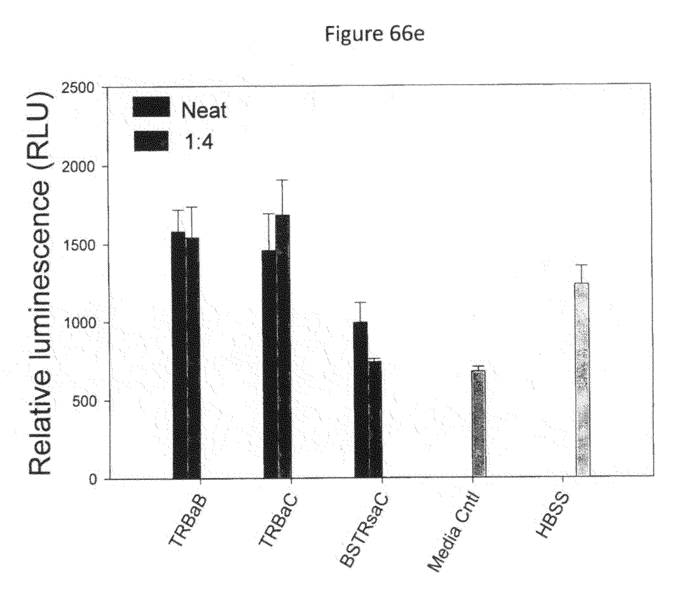

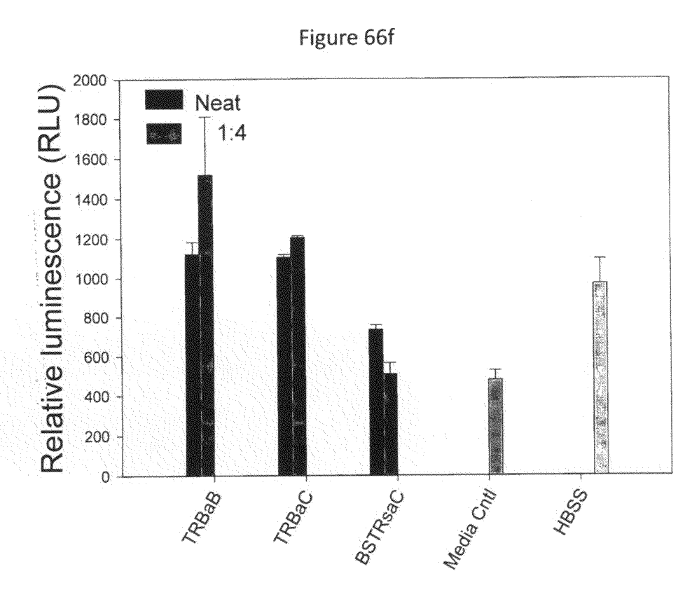

FIGS. 66a-f show that all constructs show bioactivity in the bioassay.

### MATERIALS AND METHODS

### Cell Line Development

TSH constructs were cloned into 2 separate mammalian expression vectors with different 5′ UTRs. Each construct was transfected in triplicate into CHO cells. Cells underwent a “preselection” in nucleotide free media and once stabilized further selection was done in the presence of MTX.

### Screening and Selection of Clones

Individual CHO clones were screened for TSH construct expression by ELISA and western blotting performed on serum free media. For ELISA detection was made both by a polyclonal antibody to TSH and a monoclonal antibody to TSH. For western blotting protein was identified with both a polyclonal antibody to TSH and a polyclonal antibody to TSH receptor. The polyclonal antibody to TSH detects both the alpha and beta chains although giving a higher signal for the alpha chain of TSH.

### TSH Bioassay

The TSH bioassay was performed in a CHO cell line stably expressing hTSHR and a luciferase reporter. For this assay standard TSH is normally diluted in Hank's Balanced Salt Solution (HBSS) containing BSA. This TSH/HBSS/BSA solution is added to the cells for a 6 hour incubation, followed by measurement of luciferase. For testing media from CHO cells the possibility of matrix interference was studied. To do this TSH at 0.4 μg/mL was spiked into conditioned and unconditioned media. This identified that the media matrix did have a minor inhibitory effect and thus in all assays the negative control was media. Samples were tested neat, and at 1:2, 1:4 dilutions with HBSS/BSA.

### Method for Measuring TSH Construct PK and PD

Essentially two methods will be used to measure PK and PD. This will be done in rats with no thyroid suppression and in mice where the mouse endogenous TSH has been suppressed with a T3 pellet. Rats will be used for slow release formulation evaluation. Rats will be dosed with TSH construct at 0.5 mg/kg with 6 rats per group and construct measured by ELISA for PK. 7 timepoints will be used (0, 1, 2, 4, 6, 8, 24 hours). In addition, T3 and T4 will be measured at each timepoint for PD. The mouse model has been used previously as a release test for TSH and recently modified for PK purposes. Mice are implanted with T3 pellet to suppress T4, 4-6 days later a dose of TSH or TSH construct is given at 0.4 mg/kg with 10 mice per group and measurement of T4 made at three timepoints (0, 6, 24).

### Recombinant Production of Fusion Proteins

The components of the fusion proteins were generated by PCR using primers designed to anneal to the ligand or receptor and to introduce suitable restriction sites for cloning into the target vector. The template for the PCR comprised the target gene and was obtained from IMAGE clones, cDNA libraries or from custom synthesised genes. Once the ligand and receptor genes with the appropriate flanking restriction sites had been synthesised, these were then ligated either side of the linker region in the target vector. The construct was then modified to contain the correct linker without flanking restriction sites by the insertion of a custom synthesised length of DNA between two unique restriction sites either side of the linker region, by mutation of the linker region by ssDNA modification techniques, by insertion of a primer duplex/multiplex between suitable restriction sites or by PCR modification.

Alternatively, the linker with flanking sequence, designed to anneal to the ligand or receptor domains of choice, was initially synthesised by creating an oligonucleotide duplex and this processed to generate double-stranded DNA. PCRs were then performed using the linker sequence as a “megaprimer”, primers designed against the opposite ends of the ligand and receptor to which the “megaprimer” anneals to and with the ligand and receptor as the templates. The terminal primers were designed with suitable restriction sites for ligation into the expression vector of choice.

### Expression and Purification of Fusion Proteins

Expression was carried out in a suitable system (e.g. mammalian CHO cells, E. coli) and this was dependant on the vector into which the fusion gene was generated. Expression was then analysed using a variety of methods which could include one or more of SDS-PAGE, Native PAGE, western blotting, ELISA.

Once a suitable level of expression was achieved the fusions were expressed at a larger scale to produce enough protein for purification and subsequent analysis.

Purification was carried out using a suitable combination of one or more chromatographic procedures such as ion exchange chromatography, hydrophobic interaction chromatography, ammonium sulphate precipitation, gel filtration, size exclusion and/or affinity chromatography (using nickel/cobalt-resin, antibody-immobilised resin and/or ligand/receptor-immobilised resin).

Purified protein was analysed using a variety of methods which could include one or more of Bradford's assay, SDS-PAGE, Native PAGE, western blotting, ELISA.

### Characterisation of Fusions

Denaturing PAGE, native PAGE gels and western blotting were used to analyse the fusion polypeptides and western blotting performed with antibodies non-conformationally sensitive to the fusion. Native solution state molecular weight information can be obtained from techniques such as size exclusion chromatography using a Superose G200 analytical column and analytical ultracentrifugation.

1. A fusion protein comprising thyroid stimulating hormone α [TSHα] and/or thyroid stimulating hormone β [TSHβ] wherein TSHα and/or TSHβ is optionally linked, either directly or indirectly, to the binding domain of TSH receptor.

2. A fusion protein according to claim 1 wherein TSHα and/or TSHβ is linked to said receptor binding domain and is positioned amino terminal to said receptor binding domain in said fusion protein.

3. A fusion protein according to claim 1 wherein TSHα and/or TSHβ is linked to said receptor binding domain and is positioned carboxyl-terminal to said binding domain in said fusion protein.

4. A fusion protein according to any of claims 1-3 wherein TSHα and/or TSHβ is linked to the receptor binding domain by a peptide linker.

5. A fusion protein according to claim 4 wherein said peptide linking molecule comprises at least one copy of the peptide Gly Gly Gly Gly Ser.

6. A fusion protein according to claim 5 wherein invention said peptide linking molecule comprises 1-10 copies of the peptide Gly Gly Gly Gly Ser.

7. A fusion protein according to any of claims 1-6 wherein said peptide linker molecule is modified to include at least one motif for the addition of at least one sugar moiety.

8. A fusion protein according to claim 7 wherein said peptide linker molecule comprises at least one copy of the motif (Xaa₁ Xaa₂ Xaa₃ Xaa₄ Xaa₅) wherein said motif comprises the glycosylation motif Asn-Xaa-Ser or Asn-Xaa-Thr.

9. A fusion protein according to any of claims 1-3 wherein said fusion protein does not comprise a peptide linking molecule and is a direct fusion of TSHα and/or TSHβ and the receptor binding domain.

10. A fusion protein according to claim 1 wherein said protein is selected from the group consisting of:
i) a protein comprising an amino acid sequence as represented in FIG. 4a, 4b or 4c;
ii) a protein comprising an amino acid sequence as represented in FIG. 4a, 4b or 4c that is modified by addition, or deletion or substitution of at least one amino acid residue and that modulates TSH receptor activity.

11. A fusion protein according to claim 1 wherein said protein is selected from the group consisting of:
i) a protein comprising an amino acid sequence as represented in FIG. 5a, 5b or 5c;
ii) a protein comprising an amino acid sequence as represented in FIG. 5a, 5b or 5c that is modified by addition, or deletion or substitution of at least one amino acid residue and that modulates TSH receptor activity.

12. A fusion protein according to claim 1 wherein said fprotein is selected from the group consisting of:
i) a protein comprising an amino acid sequence as represented in FIG. 6a, 6b or 6c;
ii) a protein comprising an amino acid sequence as represented in FIG. 6a, 6b or 6c that is modified by addition, or deletion or substitution of at least one amino acid residue and that modulates TSH receptor activity.

13. A fusion protein according to claim 1 wherein said protein is selected from the group consisting of:
i) a protein comprising an amino acid sequence as represented in FIG. 7a, 7b or 7c;
ii) a protein comprising an amino acid sequence as represented in FIG. 7a, 7b or 7c that is modified by addition, or deletion or substitution of at least one amino acid residue and that modulates TSH receptor activity.

14. A fusion protein according to claim 1 wherein said protein is selected from the group consisting of:
i) a protein comprising an amino acid sequence as represented in FIG. 8a, 8b or 8c;
ii) a protein comprising an amino acid sequence as represented in FIG. 8a, 8b or 8c that is modified by addition, or deletion or substitution of at least one amino acid residue and that modulates TSH receptor activity.

15. A fusion protein according to claim 1 wherein said protein is selected from the group consisting of:
i) a protein comprising an amino acid sequence as represented in FIG. 9a, 9b or 9c;
ii) a protein comprising an amino acid sequence as represented in FIG. 9a, 9b or 9c that is modified by addition, or deletion or substitution of at least one amino acid residue and that modulates TSH receptor activity.

16. A fusion protein according to claim 1 wherein said protein is selected from the group consisting of:
i) a protein comprising an amino acid sequence as represented in FIG. 10a, 10b or 10c;
ii) a protein comprising an amino acid sequence as represented in FIG. 10a, 10b or 10c that is modified by addition, or deletion or substitution of at least one amino acid residue and that modulates TSH receptor activity.

17. A fusion protein according to claim 1 wherein said protein is selected from the group consisting of:
i) a protein comprising an amino acid sequence as represented in FIG. 11a, 11b or 11c;
ii) a protein comprising an amino acid sequence as represented in FIGS. 11a, 11b or 11c that is modified by addition, or deletion or substitution of at least one amino acid residue and that modulates TSH receptor activity.

18. A fusion protein according to claim 1 wherein said protein is selected from the group consisting of:
i) a protein comprising an amino acid sequence as represented in FIG. 12.1a, 12.2a, 12b or 12c;
ii) a protein comprising an amino acid sequence as represented in FIG. 12a, 12b or 12c that is modified by addition, or deletion or substitution of at least one amino acid residue and that that modulates TSH receptor activity.

19. A fusion protein according to claim 1 wherein said protein is selected from the group consisting of:
i) a protein comprising an amino acid sequence as represented in FIG. 13.1a, 13.2a, 13b or 13c;
ii) a protein comprising an amino acid sequence as represented in FIG. 13a, 13b or 13c that is modified by addition, or deletion or substitution of at least one amino acid residue and that that modulates TSH receptor activity.

20. A fusion protein according to claim 1 wherein said fusion protein is selected from the group consisting of:
i) a protein comprising an amino acid sequence as represented in FIG. 14a, 14b or 14c;
ii) a protein comprising an amino acid sequence as represented in FIG. 14a, 14b or 14c that is modified by addition, or deletion or substitution of at least one amino acid residue and that that modulates TSH receptor activity.

21. A fusion protein according to claim 1 wherein said fusion protein is selected from the group consisting of:
i) a protein comprising an amino acid sequence as represented in FIG. 15a, 15b or 15c;
ii) a protein comprising an amino acid sequence as represented in FIG. 15a, 15b or 15c that is modified by addition, or deletion or substitution of at least one amino acid residue and that that modulates TSH receptor activity.

22. A fusion protein according to claim 1 wherein said protein is selected from the group consisting of:
i) a protein comprising an amino acid sequence as represented in FIG. 16a, 16b or 16c;
ii) a protein comprising an amino acid sequence as represented in FIG. 16a, 16b or 16c that is modified by addition, or deletion or substitution of at least one amino acid residue and that that modulates TSH receptor activity.

23. A fusion protein according to claim 1 wherein said protein is selected from the group consisting of:
iii) a protein comprising an amino acid sequence as represented in FIG. 17a, 17b or 17c;
iv) a protein comprising an amino acid sequence as represented in FIG. 17a, 17b or 17c that is modified by addition, or deletion or substitution of at least one amino acid residue and that that modulates TSH receptor activity.

24. A fusion protein according to claim 1 wherein said protein is selected from the group consisting of:
i) a protein comprising an amino acid sequence as represented in FIG. 18a, 18b or 18c;
ii) a protein comprising an amino acid sequence as represented in FIG. 18a, 18b or 18c that is modified by addition, or deletion or substitution of at least one amino acid residue and that that modulates TSH receptor activity.

25. A fusion protein according to claim 1 wherein said protein is selected from the group consisting of:
i) a protein comprising an amino acid sequence as represented in FIG. 19a, 19b or 19c;
ii) a protein comprising an amino acid sequence as represented in FIG. 19a, 19b or 19c that is modified by addition, or deletion or substitution of at least one amino acid residue and that that modulates TSH receptor activity.

26. A fusion protein according to claim 1 wherein said protein is selected from the group consisting of:
i) a protein comprising an amino acid sequence as represented in FIG. 20a, 20b or 20c;
ii) a protein comprising an amino acid sequence as represented in FIG. 20a, 20b or 20c that is modified by addition, or deletion or substitution of at least one amino acid residue and that that modulates TSH receptor activity.

27. A fusion protein according to claim 1 wherein said protein is selected from the group consisting of:
i) a protein comprising an amino acid sequence as represented in FIG. 21a, 21b or 21c;
ii) a protein comprising an amino acid sequence as represented in FIG. 21a, 21b or 21c that is modified by addition, or deletion or substitution of at least one amino acid residue and that that modulates TSH receptor activity.

28. A fusion protein according to claim 1 wherein said fusion protein is selected from the group consisting of:
i) a protein comprising an amino acid sequence as represented in FIG. 22a, 22b or 22c;
ii) a protein comprising an amino acid sequence as represented in FIG. 22a, 22b or 22c that is modified by addition, or deletion or substitution of at least one amino acid residue and that that modulates TSH receptor activity.

29. A fusion protein according to claim 1 wherein said protein is selected from the group consisting of:
i) a protein comprising an amino acid sequence as represented in FIG. 23a, 23b or 23c;
ii) a protein comprising an amino acid sequence as represented in FIG. 23a, 23b or 23c that is modified by addition, or deletion or substitution of at least one amino acid residue and that that modulates TSH receptor activity.

30. A fusion protein according to claim 1 wherein said protein is selected from the group consisting of:
i) a protein comprising an amino acid sequence as represented in FIG. 24a, 24b or 24c;
ii) a protein comprising an amino acid sequence as represented in FIG. 24a, 24b or 24c that is modified by addition, or deletion or substitution of at least one amino acid residue and that that modulates TSH receptor activity.

31. A fusion protein according to claim 1 wherein said protein is selected from the group consisting of:
i) a protein comprising an amino acid sequence as represented in FIG. 25a, 25b or 25c;
ii) a protein comprising an amino acid sequence as represented in FIG. 25a, 25b or 25c that is modified by addition, or deletion or substitution of at least one amino acid residue and that that modulates TSH receptor activity.

32. A fusion protein according to claim 1 wherein said protein is selected from the group consisting of:
i) a protein comprising an amino acid sequence as represented in FIG. 26a, 26b or 26c;
ii) a protein comprising an amino acid sequence as represented in FIG. 26a, 26b or 26c that is modified by addition, or deletion or substitution of at least one amino acid residue and that that modulates TSH receptor activity.

33. A fusion protein according to claim 1 wherein said protein is selected from the group consisting of:
i) a protein comprising an amino acid sequence as represented in FIG. 27a, 27b or 27c;
ii) a protein comprising an amino acid sequence as represented in FIG. 27a, 27b or 27c that is modified by addition, or deletion or substitution of at least one amino acid residue and that that modulates TSH receptor activity.

34. A fusion protein according to claim 1 wherein said protein is selected from the group consisting of:
i) a protein comprising an amino acid sequence as represented in FIG. 28a, 28b or 28c;
ii) a protein comprising an amino acid sequence as represented in FIG. 28a, 28b or 28c that is modified by addition, or deletion or substitution of at least one amino acid residue and that that modulates TSH receptor activity.

35. A fusion protein according to claim 1 wherein said protein is selected from the group consisting of:
i) a protein comprising an amino acid sequence as represented in FIG. 29a, 29b or 29c;
ii) a protein comprising an amino acid sequence as represented in FIG. 29a, 29b or 29c that is modified by addition, or deletion or substitution of at least one amino acid residue and that that modulates TSH receptor activity.

36. A fusion protein according to claim 1 wherein said protein is selected from the group consisting of:
i) a protein comprising an amino acid sequence as represented in FIG. 30a, 30b or 30c;
ii) a protein comprising an amino acid sequence as represented in FIG. 30a, 30b or 30c that is modified by addition, or deletion or substitution of at least one amino acid residue and that that modulates TSH receptor activity.

37. A fusion protein according to claim 1 wherein said protein is selected from the group consisting of:
i) a protein comprising an amino acid sequence as represented in FIG. 31a, 31b or 31c;
ii) a protein comprising an amino acid sequence as represented in FIG. 31a, 31b or 31c that is modified by addition, or deletion or substitution of at least one amino acid residue and that that modulates TSH receptor activity.

38. A fusion protein according to claim 1 wherein said fusion protein is selected from the group consisting of:
i) a protein comprising an amino acid sequence as represented in FIG. 32a, 32b or 32c;
ii) a protein comprising an amino acid sequence as represented in FIG. 32a, 32b or 32c that is modified by addition, or deletion or substitution of at least one amino acid residue and that that modulates TSH receptor activity.

39. A fusion protein according to claim 1 wherein said protein is selected from the group consisting of:
i) a protein comprising an amino acid sequence as represented in FIG. 33a, 33b or 33c;
ii) a protein comprising an amino acid sequence as represented in FIG. 33a, 33b or 33c that is modified by addition, or deletion or substitution of at least one amino acid residue and that that modulates TSH receptor activity.

40. A fusion protein according to claim 1 wherein said protein is selected from the group consisting of:
i) a protein comprising an amino acid sequence as represented in FIG. 34a, 34b or 34c;
ii) a protein comprising an amino acid sequence as represented in FIG. 34a, 34b or 34c that is modified by addition, or deletion or substitution of at least one amino acid residue and that that modulates TSH receptor activity.

41. A fusion protein according to claim 1 wherein said protein is selected from the group consisting of:
i) a protein comprising an amino acid sequence as represented in FIG. 35a, 35b or 35c;
ii) a protein comprising an amino acid sequence as represented in FIG. 35a, 35b or 35c that is modified by addition, or deletion or substitution of at least one amino acid residue and that that modulates TSH receptor activity.

42. A fusion protein according to claim 1 wherein said protein is selected from the group consisting of:
i) a protein comprising an amino acid sequence as represented in FIG. 36a, 36b or 36c;
ii) a protein comprising an amino acid sequence as represented in FIG. 36a, 36b or 36c that is modified by addition, or deletion or substitution of at least one amino acid residue and that that modulates TSH receptor activity.

43. A fusion protein according to claim 1 wherein said protein is selected from the group consisting of:
i) a protein comprising an amino acid sequence as represented in FIG. 37a, 37b or 37c;
ii) a protein comprising an amino acid sequence as represented in FIG. 37a, 37b or 37c that is modified by addition, or deletion or substitution of at least one amino acid residue and that that modulates TSH receptor activity.

44. A fusion protein according to claim 1 wherein said protein is selected from the group consisting of:
i) a protein comprising an amino acid sequence as represented in FIG. 38a, 38b or 38c;
ii) a protein comprising an amino acid sequence as represented in FIG. 38a, 38b or 38c that is modified by addition, or deletion or substitution of at least one amino acid residue and that that modulates TSH receptor activity.

45. A fusion protein according to claim 1 wherein said protein is selected from the group consisting of:
i) a protein comprising an amino acid sequence as represented in FIG. 39a, 39b or 39c;
ii) a protein comprising an amino acid sequence as represented in FIG. 39a, 39b or 39c that is modified by addition, or deletion or substitution of at least one amino acid residue and that that modulates TSH receptor activity.

46. A fusion protein according to claim 1 wherein said protein is selected from the group consisting of:
i) a protein comprising an amino acid sequence as represented in FIG. 40a, 40b or 40c;
ii) a protein comprising an amino acid sequence as represented in FIG. 40a, 40b or 40c that is modified by addition, or deletion or substitution of at least one amino acid residue and that that modulates TSH receptor activity.

47. A fusion protein according to claim 1 wherein said protein is selected from the group consisting of:
i) a protein comprising an amino acid sequence as represented in FIG. 41a, 41b or 41c;
ii) a protein comprising an amino acid sequence as represented in FIG. 41a, 41b or 41c that is modified by addition, or deletion or substitution of at least one amino acid residue and that that modulates TSH receptor activity.

48. A fusion protein according to claim 1 wherein said protein is selected from the group consisting of:
i) a protein comprising an amino acid sequence as represented in FIG. 42a, 42b or 42c;
ii) a protein comprising an amino acid sequence as represented in FIG. 42a, 42b or 42c that is modified by addition, or deletion or substitution of at least one amino acid residue and that that modulates TSH receptor activity.

49. A fusion protein according to claim 1 wherein said protein is selected from the group consisting of:
i) a protein comprising an amino acid sequence as represented in FIG. 43a, 43b or 43c;
ii) a protein comprising an amino acid sequence as represented in FIG. 43a, 43b or 43c that is modified by addition, or deletion or substitution of at least one amino acid residue and that that modulates TSH receptor activity.

50. A fusion protein according to claim 1 wherein protein is selected from the group consisting of:
i) a protein comprising an amino acid sequence as represented in FIG. 44a, 44b or 44c;
ii) a protein comprising an amino acid sequence as represented in FIG. 44a, 44b or 44c that is modified by addition, or deletion or substitution of at least one amino acid residue and that that modulates TSH receptor activity.

51. A fusion protein according to claim 1 wherein said protein is selected from the group consisting of:
i) a protein comprising an amino acid sequence as represented in FIG. 45a, 45b or 45c;
ii) a protein comprising an amino acid sequence as represented in FIG. 45a, 45b or 45c that is modified by addition, or deletion or substitution of at least one amino acid residue and that that modulates TSH receptor activity.

52. A fusion protein according to claim 1 wherein said protein is selected from the group consisting of:
i) a protein comprising an amino acid sequence as represented in FIG. 46a, 46b or 46c;
ii) a protein comprising an amino acid sequence as represented in FIG. 46a, 46b or 46c that is modified by addition, or deletion or substitution of at least one amino acid residue and that that modulates TSH receptor activity.

53. A fusion protein according to claim 1 wherein said protein is selected from the group consisting of:
i) a protein comprising an amino acid sequence as represented in FIG. 47a, 47b or 47c;
ii) a protein comprising an amino acid sequence as represented in FIG. 47a, 47b or 47c that is modified by addition, or deletion or substitution of at least one amino acid residue and that that modulates TSH receptor activity.

54. A fusion protein according to claim 1 wherein said protein is selected from the group consisting of:
i) a protein comprising an amino acid sequence as represented in FIG. 48a, 48b or 48c;
ii) a protein comprising an amino acid sequence as represented in FIG. 48a, 48b or 48c that is modified by addition, or deletion or substitution of at least one amino acid residue and that that modulates TSH receptor activity.

55. A fusion protein according to claim 1 wherein said protein is selected from the group consisting of:
i) a protein comprising an amino acid sequence as represented in FIG. 49a, 49b or 49c;
ii) a protein comprising an amino acid sequence as represented in FIG. 49a, 49b or 49c that is modified by addition, or deletion or substitution of at least one amino acid residue and that that modulates TSH receptor activity.

56. A fusion protein according to claim 1 wherein said protein is selected from the group consisting of:
i) a protein comprising an amino acid sequence as represented in FIG. 50a, 50b or 50c;
ii) a protein comprising an amino acid sequence as represented in FIG. 50a, 50b or 50c that is modified by addition, or deletion or substitution of at least one amino acid residue and that that modulates TSH receptor activity.

57. A fusion protein according to claim 1 wherein said protein is selected from the group consisting of:
i) a protein comprising an amino acid sequence as represented in FIG. 51a, 51b or 51c;
ii) a protein comprising an amino acid sequence as represented in FIG. 51a, 51b or 51c that is modified by addition, or deletion or substitution of at least one amino acid residue and that that modulates TSH receptor activity.

58. A fusion protein according to claim 1 wherein said protein is selected from the group consisting of:
i) a protein comprising an amino acid sequence as represented in FIG. 52a, 52b or 52c;
ii) a protein comprising an amino acid sequence as represented in FIG. 52a, 52b or 52c that is modified by addition, or deletion or substitution of at least one amino acid residue and that that modulates TSH receptor activity.

59. A fusion protein according to claim 1 wherein said protein is selected from the group consisting of:
i) a protein comprising an amino acid sequence as represented in FIG. 53a, 53b or 53c;
ii) a protein comprising an amino acid sequence as represented in FIG. 53a, 53b or 53c that is modified by addition, or deletion or substitution of at least one amino acid residue and that that modulates TSH receptor activity.

60. A fusion protein according to claim 1 wherein said fusion protein comprises of TSHα and TSHβ linked in tandem.

61. A fusion protein according to claim 60 wherein said fusion protein comprises first and second TSHα and TSHβ polypeptides.

62. A fusion protein according to claim 61 wherein said first and second TSHα and TSHβ polypeptides are linked in tandem wherein said fusion protein comprises:
i) TSHβ-TSHα-TSHβ-TSHα; or
ii) TSHα-TSHβ-TSHβ-TSHα; or
iii) TSHβ-TSHα-TSHα-TSHβ; or
iv) TSHα-TSHβ-TSHα-TSHβ.

63. A fusion protein according to any of claims 60-62 wherein said protein is selected from the group consisting of:
i) a protein comprising an amino acid sequence as represented in FIG. 54;
ii) a protein comprising an amino acid sequence as represented in FIG. 54 that is modified by addition, or deletion or substitution of at least one amino acid residue and that that modulates TSH receptor activity.

64. A fusion protein according to any of claims 60-62 wherein said protein is selected from the group consisting of:
i) a protein comprising an amino acid sequence as represented in FIG. 55;
ii) a protein comprising an amino acid sequence as represented in FIG. 55 that is modified by addition, or deletion or substitution of at least one amino acid residue and that that modulates TSH receptor activity.

65. A fusion protein according to any of claims 60-62 wherein said protein is selected from the group consisting of:
i) a protein comprising an amino acid sequence as represented in FIG. 56;
ii) a protein comprising an amino acid sequence as represented in FIG. 56 that is modified by addition, or deletion or substitution of at least one amino acid residue and that that modulates TSH receptor activity.

66. A fusion protein according to any of claims 60-62 wherein said protein is selected from the group consisting of:
i) a protein comprising an amino acid sequence as represented in FIG. 57;
ii) a protein comprising an amino acid sequence as represented in FIG. 57 that is modified by addition, or deletion or substitution of at least one amino acid residue and that that modulates TSH receptor activity.

67. A fusion protein according to any of claims 60-62 wherein said protein is selected from the group consisting of:
i) a protein comprising an amino acid sequence as represented in FIG. 58;
ii) a protein comprising an amino acid sequence as represented in FIG. 58 that is modified by addition, or deletion or substitution of at least one amino acid residue and that that modulates TSH receptor activity.

68. A fusion protein according to any of claims 60-62 wherein said protein is selected from the group consisting of:
i) a protein comprising an amino acid sequence as represented in FIG. 59;
ii) a protein comprising an amino acid sequence as represented in FIG. 59 that is modified by addition, or deletion or substitution of at least one amino acid residue and that that modulates TSH receptor activity.

69. A fusion protein according to any of claims 60-62 wherein said protein is selected from the group consisting of:
i) a protein comprising an amino acid sequence as represented in FIG. 60;
ii) a protein comprising an amino acid sequence as represented in FIG. 60 that is modified by addition, or deletion or substitution of at least one amino acid residue and that that modulates TSH receptor activity.

70. A fusion protein according to any of claims 60-62 wherein said fusion protein is selected from the group consisting of:
i) a protein comprising an amino acid sequence as represented in FIG. 61;
ii) a protein comprising an amino acid sequence as represented in FIG. 61 that is modified by addition, or deletion or substitution of at least one amino acid residue and that that modulates TSH receptor activity.

71. A nucleic acid molecule that encodes a fusion protein according to any of claims 1-70.

72. A vector comprising a nucleic acid molecule according to claim 71.

73. A cell transfected or transformed with a nucleic acid molecule or vector according to claim 71 or 72.

74. A pharmaceutical composition comprising a polypeptide according to any of claims 1-70 including an excipient or carrier.

75. A pharmaceutical composition according to claim 74 wherein said polypeptide is combined with a further therapeutic agent.

76. A method to treat a subject suffering from thyroid cancer comprising administering an effective amount of a TSH fusion protein according to any of claims 1-70.

77. A method to treat a subject suffering from multinodular goiter comprising administering an effective amount of a TSH fusion protein according to any of claims 1-70.

78. A method to treat a subject suffering from thyrotoxicosis comprising administering an effective amount of a TSH fusion protein according to any of claims 1-70.

79. A method to treat a subject suffering from secondary hypothyroidism comprising administering an effective amount of a TSH fusion protein according to any of claims 1-70.

80. A method to treat a subject suffering from osteoporosis comprising administering an effective amount of a TSH fusion protein according to any of claims 1-70.
